(19) United States
(12) Patent Application Publication
(10) Pub. No.: US 2012/0089118 A1
(43) Pub. Date: Apr. 12, 2012

(54) DELIVERY OF BISPHOSPHONATES BY MICROINJECTION SYSTEMS

(75) Inventors: Anthony P. Deasey, Severna Park, MD (US); Patrick Frankham, Rosemere (CA); Wolfgang Renz, Beaconsfield (CA); Thomas Lang, Marshfield, MA (US)

(73) Assignees: A Nevada Corporation; LANCO Biosciences, Inc, Las Vegas, NV (US)

(21) Appl. No.: 13/269,491

(22) Filed: Oct. 7, 2011

Related U.S. Application Data
(60) Provisional application No. 61/391,501, filed on Oct. 8, 2010.
(60) Provisional application No. 61/419,727, filed on Dec. 3, 2010.

(51) Int. Cl. A61M 5/00

(52) U.S. Cl. 604/506; 604/173

(57) ABSTRACT

A system for the administration of a bisphosphonate to a subject comprises a bisphosphonate formulation and a microinjection device. In an embodiment, the bisphosphonate formulation includes ibandronate sodium. In some cases, the bisphosphonate formulation includes an excipient. The bisphosphonate formulation may be delivered to the subject subcutaneously, transdermally or intradermally.

### CROSS-REFERENCE

This applications claims priority to U.S. Provisional Patent Application Ser. No. 61/391,501, filed Oct. 8, 2010, and U.S. Provisional Patent Application Ser. No. 61/419,727, filed Dec. 3, 2010, which are entirely incorporated herein by reference.

### BACKGROUND OF THE INVENTION

Osteoporosis is a disease of the bone that can lead to an increased risk of bone fracture. In osteoporosis, the bone mineral density is reduced, bone microarchitecture is disrupted, and the amount and variety of proteins in bone is altered.

The underlying mechanism in all cases of osteoporosis is an imbalance between bone resorption and bone formation. In normal bone, there is constant matrix remodeling of bone; up to about 10% of all bone mass can be undergoing remodeling at any point in time. The process takes place in bone multicellular units (BMUs). See, e.g., H. M. Frost and C. C. Thomas, “Bone Remodeling Dynamics”, Springfield, Ill. (1963). Bone is resorbed by osteoclast cells (which derive from the bone marrow), after which new bone is deposited by osteoblast cells.

Mechanisms by which osteoporosis develops include inadequate peak bone mass (the skeleton develops insufficient mass and strength during growth), excessive bone resorption and inadequate formation of new bone during remodeling. Interplay of these mechanisms can underlie the development of fragile bone tissue. See L. Raisz, “Pathogenesis of osteoporosis: concepts, conflicts, and prospects,” J. Clin. Invest. 115 (12), 3318-25 (2005).

### SUMMARY OF THE INVENTION

In an aspect of the invention, a system comprising a microinjection device and a bisphosphonate formulation is provided. The microinjection device comprises a microneedle array having one or more hollow tips for delivering a bisphosphonate formulation, a housing having the microneedle array and a skin-contacting face defining an opening that can be positioned at or adjacent to a target site, and a driver for moving the microneedle array toward the target site. In an embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0. In another embodiment, the microinjection device is configured for subcutaneous, transdermal or intradermal delivery of the bisphosphonate formulation to a subject.

In another aspect of the invention, a method for delivering a bisphosphonate formulation to a subject comprises providing a microinjection device comprising a microneedle array and a bisphosphonate formulation, and delivering the bisphosphonate formulation to the subject with the aid of the microinjection device. In an embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0. In another embodiment, the bisphosphonate formulation is delivered to the subject between three times a year and six times a year. In another embodiment, the bisphosphonate formulation is delivered to the subject four times a year. In another embodiment, the bisphosphonate formulation is delivered to the subject at least once a week. In another embodiment, the bisphosphonate formulation is delivered to the subject at least twice a week. In another embodiment, the bisphosphonate formulation is delivered to the subject at least once a month. In another embodiment, the bisphosphonate formulation is delivered to the subject for a duration of time between about 1 second and 10 minutes. In another embodiment, the bisphosphonate formulation is delivered to the subject for a duration of time between about 10 seconds and 1 minute.

In another aspect of the invention, a method for treating bone disease is provided, the method comprising using a microinjection device comprising a microneedle array and a bisphosphonate formulation to administer to a subject the bisphosphonate formulation. In an embodiment, the bisphosphonate formulation is administered to the subject between three times a year and six times a year. In another embodiment, the bisphosphonate formulation is administered to the subject four times a year. In another embodiment, the bisphosphonate formulation is administered to the subject at least once a week. In another embodiment, the bisphosphonate formulation is administered to the subject at least twice a week. In another embodiment, the bisphosphonate formulation is administered to the subject at least once a month. In another embodiment, the bisphosphonate formulation is delivered to the subject for a duration of time between about 1 second and 10 minutes. In another embodiment, the bisphosphonate formulation is delivered to the subject for a duration of time between about 10 seconds and 1 minute. In another embodiment, the bisphosphonate formulation is delivered to the subject transdermally. In another embodiment, the bisphosphonate formulation is delivered to the subject intradermally. In another embodiment, the bisphosphonate formulation comprises ibandronate, ibandronate sodium, ibandronate monosodium salt monohydrate, or other pharmaceutically acceptable salt of ibandronate ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a system is provided comprising an application device and a bisphosphonate formulation. The application device comprises a housing having a skin-contacting face defining an opening that can be positioned at a target site, the housing having a microneedle array. The application device also comprises an impactor for impacting the microneedle array and accelerating the microneedle array toward the target site, the microneedle array configured to deliver a bisphosphonate formulation to the subject. The impactor is configured to move along a substantially arcuate path to move the microneedle array toward the target site. In an embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a microinjection device configured to deliver a bisphosphonate formulation to a subject is provided. In an embodiment, the microinjection device is configured to deliver to a subject a bisphosphonate formulation comprising ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a microinjection device for subcutaneous, transdermal or intradermal delivery of a bisphosphonate to a subject is provided, comprising a microneedle array for delivering a bisphosphonate to a subject, and one or more chambers in fluid communication with the microneedle array, the one or more chambers configured to hold a bisphosphonate formulation. In an embodiment, the microneedle array comprises microneedles having hollow tips. In another embodiment, at least one of the one or more chambers includes a bisphosphonate formulation. In another embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a system for the administration of a bisphosphonate to a subject is provided. The system comprises a bisphosphonate formulation and a microinjection device. In an embodiment, the bisphosphonate formulation is provided in the microinjection device. In another embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation comprises sodium chloride, glacial acetic acid, sodium acetate and water. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a system for applying a microneedle array to a subject's skin comprises a bisphosphonate formulation and a housing having a skin-contacting face defining an opening that can be positioned at a target site. The housing includes a microneedle array. The system also includes an impactor for impacting the microneedle array and accelerating the microneedle array toward the target site, the microneedle array configured to deliver a bisphosphonate formulation to the subject. The impactor is configured to move along a substantially arcuate path to move the microneedle array toward the target site. In an embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a system for subcutaneous, transdermal or intradermal delivery of a bisphosphonate to a subject comprises a bisphosphonate formulation, a microneedle array for delivering the bisphosphonate formulation to a subject, and one or more chambers in fluid communication with the microneedle array, the one or more chambers configured to hold the bisphosphonate formulation. In an embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a system for delivering a bisphosphonate formulation to a subject comprises a bisphosphonate formulation, a microneedle array having one or more hollow tips for delivering the bisphosphonate formulation, a housing having the microneedle array and a skin-contacting face defining an opening that can be positioned at or adjacent to a target site, and a driver for moving the microneedle array toward the target site. In an embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0. In another embodiment, the bisphosphonate formulation comprises sodium chloride, glacial acetic acid, sodium acetate and water.

In another aspect of the invention, a microinjection device comprising a hollow microneedle array and a bisphosphonate formulation is provided. The microinjection device is configured to deliver the bisphosphonate formulation to a subject. In an embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0. In another embodiment, the bisphosphonate formulation comprises sodium chloride, glacial acetic acid, sodium acetate and water.

In another aspect of the invention, a method for subcutaneous, transdermal or intradermal delivery of a bisphosphonate formulation to a subject comprises administering the bisphosphonate formulation to the subject with ion pairs, coacervates, vesicles, liposomes, or particles. In an embodiment, particles are used to administer the bisphosphonate formulation. In another embodiment, the particles are administered to a subject's skin at a high velocity. In another embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation is administered to the subject between three times a year and six times a year. In another embodiment, the bisphosphonate formulation is administered to the subject four times a year. In another embodiment, the bisphosphonate formulation is administered to the subject at least once a week. In another embodiment, the bisphosphonate formulation is administered to the subject at least twice a week. In another embodiment, the bisphosphonate formulation is administered to the subject at least once a month. In another embodiment, the bisphosphonate formulation is administered to the subject for a duration of time between about 1 second and 10 minutes. In another embodiment, the bisphosphonate formulation is administered to the subject for a duration of time between about 10 seconds and 1 minute. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In another aspect of the invention, a method for subcutaneous, transdermal or intradermal delivery of a bisphosphonate formulation to a subject comprises administering the bisphosphonate formulation to the subject by microneedle injection, hydration, ablation of the subjects skin, follicular delivery, ultrasound, iontophoresis or electroporation. In an embodiment, the bisphosphonate formulation is administered to a subject by microneedle injection. In another embodiment, the bisphosphonate formulation is administered to a subject by iontophoresis. In another embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0. In another embodiment, the bisphosphonate formulation is administered to the subject between three times a year and six times a year. In another embodiment, the bisphosphonate formulation is administered to the subject four times a year. In another embodiment, the bisphosphonate formulation is administered to the subject at least once a week. In another embodiment, the bisphosphonate formulation is administered to the subject at least twice a week. In another embodiment, the bisphosphonate formulation is administered to the subject at least once a month. In another embodiment, the bisphosphonate formulation is administered to the subject for a duration of time between about 1 second and 10 minutes. In another embodiment, the bisphosphonate formulation is administered to the subject for a duration of time between about 10 seconds and 1 minute.

In another aspect, a system for delivering a bisphosphonate formulation comprises a bisphosphonate formulation having an excipient, and a microinjection device configured to deliver the bisphosphonate formulation. In an embodiment, the microinjection device is configured to deliver at least about 1 mg, or 2 mg, or 3 mg of bisphosphonate in 3 mL of the bisphosphonate formulation. In another embodiment, the bisphosphonate formulation comprises ibandronate or a pharmaceutically acceptable derivative of ibandronate. In another embodiment, the excipient includes one or more of sodium chloride, glacial acetic acid, sodium acetate and water. In another embodiment, the excipient is a pharmaceutically acceptable excipient. In another embodiment, the bisphosphonate formulation comprises ibandronate, ibandronate sodium, ibandronate monosodium salt monohydrate, or other pharmaceutically acceptable salt of ibandronate. In another embodiment, the bisphosphonate formulation has a bisphosphonate concentration between about 3.0 mg bisphosphonate in 3 mL and 3.5 mg in 3 mL. In another embodiment, the bisphosphonate formulation has a bisphosphonate concentration between about 3.3 mg bisphosphonate in 3 mL and 3.4 mg in 3 mL. In another embodiment, the bisphosphonate formulation has a pH between about 2.5 and 5.5. In another embodiment, the bisphosphonate formulation has a pH between about 3.0 and 4.5. In another embodiment, the bisphosphonate formulation has a pH between about 2.5 and 2.9. In another embodiment, the bisphosphonate formulation has a pH between about 4.6 and 5.0.

In some embodiments, bisphosphonate formulations for use with systems, devices or methods above, alone or in combination, comprise medronate, oxidronate, etidronate, clodronate, tiludronate, pamidronate, neridronate, olpadronate, alendronate, ibandronate, risedronate, zoledronate or a pharmaceutically acceptable salt or derivative thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a perspective view of a microinjection device having a microneedle assembly, in accordance with an embodiment of the invention; FIG. 1B is a perspective side view of an array of microneedles, in accordance with an embodiment of the invention;

FIG. 2 is a schematic cross-sectional side view of a microinjection device having an array of microneedles, in accordance with an embodiment of the invention;

FIG. 3 is a schematic cross-sectional side view of a portion of the microinjection device of FIG. 2, in accordance with an embodiment of the invention;

FIG. 4 is a schematic perspective side view of a microneedle device comprising a patch, in accordance with an embodiment of the invention;

FIG. 5A is a perspective side view of an array of microneedles, in accordance with an embodiment of the invention. FIG. 5B is a cross-sectional side view of a microneedle in the array of FIG. 5A, in accordance with an embodiment of the invention;

FIG. 6 is a schematic side view of a microneedle application device, in accordance with an embodiment of the invention;

FIG. 7 is a schematic cross sectional side view of the microneedle application device of FIG. 6, in accordance with an embodiment of the invention;

FIG. 8 is a schematic cross sectional side view of a collar of the microneedle application device of FIGS. 6 and 7, in accordance with an embodiment of the invention;

FIG. 9A is a schematic perspective view of an applicator device having peelable seals, in accordance with an embodiment of the invention. FIG. 9B is a schematic perspective view of the applicator of FIG. 9A with the peelable seals removed, in accordance with an embodiment of the invention. FIG. 9C is a schematic cross-sectional view of the applicator of FIGS. 9A and 9B in a loaded position, in accordance with an embodiment of the invention. FIG. 9D is a schematic cross-sectional view of the applicator of FIGS. 9A and 9B in a partially released position, in accordance with an embodiment of the invention. FIG. 9E is a schematic cross-sectional view of the applicator of FIGS. 9A and 9B in a position where a microneedle array can contact a target surface, in accordance with an embodiment of the invention. FIG. 9F is a schematic cross-sectional view of the applicator of FIGS. 9A and 9B being removed from a microneedle array that has been deployed onto a target surface, in accordance with an embodiment of the invention;

FIG. 10 is a schematic cross-sectional side view of an applicator device, in accordance with an embodiment of the invention;

FIG. 11 is a schematic perspective view of a portion of the applicator device of FIG. 10, in accordance with an embodiment of the invention;

FIG. 12 is a schematic perspective view of an applicator device having a patch, in accordance with an embodiment of the invention; and

FIG. 13 is a schematic partial cross-sectional side view of a microneedle array cartridge mounted on an applicator device, in accordance with an embodiment of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention.

The term “bisphosphonate,” as used herein, refers to any chemical or compound having two phosphonate (PO₃) groups. In an embodiment, bisphosphonates can include phosphonic acids and derivatives of phosphonic acids. Examples of bisphosphonates include medronate, oxidronate, etidronate, clodronate, tiludronate, pamidronate, neridronate, olpadronate, alendronate, ibandronate, risedronate, zoledronate and pharmaceutically acceptable salts or derivatives thereof. A pharmaceutically acceptable salt can include, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Examples of bisphosphonates include dichloromethylene-diphosphonate, aminohydroxypropane-diphosphonate, aminohydroxybutane-diphosphonate, aminohydroxypentane-diphosphonate or aminohydroxyhexane-diphosphonate. In an embodiment, bisphosphonates include ibandronate sodium (3-(N-methyl-N-pentyl)amino-1-hydroxypropane-1,1-diphosphonic acid, monosodium salt, monohydrate, having molecular formula C₉H₂₂NO₇P₂Na*H₂O) or other pharmaceutically acceptable salts of ibandronate. In an embodiment, a bisphosphonate can be ibandronate sodium or ibandronate monosodium salt monohydrate. In other examples, bisphosphonates include compounds having two PO₃ (phosphonate) groups covalently linked to carbon.

The terms “transdermal” and “transdermally,” as used herein, can refer to transdermal drug delivery. In an embodiment, transdermal drug delivery can include delivering a drug or formulation to a subject across the subject's skin. In another embodiment, transdermal drug delivery can include delivering a drug or formulation to a subject across the subject's skin and into a blood vessel.

The terms “intradermal” and “intradermally,” as used herein, can refer to intradermal drug delivery. In an embodiment, intradermal drug delivery can include delivering a drug or formulation to a subject in or into the subject's skin.

The term “subcutaneous injection,” as used herein, can refer to an injection that is administered as a bolus into the subcutis, i.e., the layer of skin directly below the dermis and epidermis (collectively referred to as the cutis).

Bisphosphonate compounds provided herein can be used to treat osteoporosis and similar diseases. Bone has constant turnover, and is kept in balance (under steady state or homeostatic conditions) by osteoblasts and osteoclasts, which are cells that generate and digest bone, respectively. Bisphosphonates can aid in preventing the loss of bone mass by inhibiting the digestion of bone by osteoclasts. Osteoclasts also have constant turnover and can destroy themselves by apoptosis. Bisphosphonates can encourage osteoclasts to undergo apoptosis. The uses of bisphosphonates can include the prevention and treatment of osteoporosis, osteitis deformans, bone metastasis, multiple myeloma, primary hyperparathyroidism, osteogenesis imperfecta and other conditions that feature bone fragility. In some cases, bisphosphonates may be used to treat skeletal metastasis in several forms of cancer, such as breast cancer.

In some embodiments, injection microinjection devices, systems and methods are provided for delivering bisphosphonate compounds (also “bisphosphonates” herein) to a subject.

### Bisphosphonate Compounds and Formulations

In an aspect of the invention, bisphosphonate compounds are provided. Bisphosphonate compounds can be included in formulations that can include other compounds or agents, such as pharmacological agents. Bisphosphonate formulations can be suited for reducing, if not eliminating, bone loss. Formulations (or injection solutions) can be stable upon storage in a container. In an embodiment, a bisphosphonate formulation including ibandronate salt, such as, e.g., ibandronate sodium, is provided.

In an embodiment, a bisphosphonate formulation is provided including ibandronate sodium, the ibandronate sodium having the following structure:

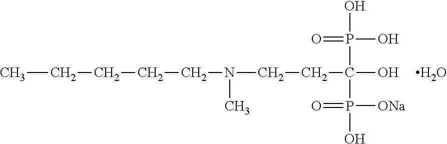

The ibandronate sodium formulation can be configured for subcutaneous, intradermal, or transdermal application to a subject (e.g., patient). The ibandronate sodium formulation can be delivered to a subject with the aid of microinjection or microneedle devices, as described below.

Injection solutions having bisphosphonates can be filled into primary packaging, such as injection or microinjection apparatuses, as described below. Bisphosphonate compounds and formulations can be administered to one or more subjects with the aid of injection and microinjection apparatuses provided herein.

In an embodiment, a bisphosphonate formulation comprises an aqueous solution having at least one diphosphonic acid or physiologically acceptable salt or ester thereof as the active substance (or compound). In an embodiment, the solution having the diphosphonic acid is stabilized by having a pH value between about 3.0 and 4.5. In another embodiment, the solution having the diphosphonic acid is stabilized by having one or more polyethylene glycols and a pH value of between about 3.0 and 4.2.

In an embodiment, a bisphosphonate formulation has a pH between about 2.5 and 5.5. In another embodiment, a bisphosphonate formulation has a pH between about 3.0 and 5.0. In yet another embodiment, a bisphosphonate formulation has a pH between about 3.5 and 4.5. In still another embodiment, a bisphosphonate formulation has a pH between about 4.0 and 4.5.

In an embodiment, a bisphosphonate formulation has a pH of about 2.5, or 2.6, or 2.7, or 2.8, or 2.9, or 3.0, or 3.1, or 3.2, or 3.3, or 3.4, or 3.5, or 3.6, or 3.7, or 3.8, or 3.9, or 4.0, or 4.1, or 4.2, or 4.3, or 4.4, or 4.5, or 4.6, or 4.7, or 4.8, or 4.9, or 5.0. In another embodiment, a bisphosphonate formulation has a pH between about 2.5 and 3.5, or between about 2.5 and 2.9. In yet another embodiment, a bisphosphonate formulation has a pH between about 4.6 and 5.5, or between about 4.6 and 5.0.

In an embodiment, an ibandronate-containing formulation has a pH between about 2.5 and 5.5. In another embodiment, an ibandronate-containing formulation has a pH between about 3.0 and 5.0. In yet another embodiment, an ibandronate-containing formulation has a pH between about 3.5 and 4.5. In still another embodiment, an ibandronate-containing formulation has a pH between about 4.0 and 4.5.

In an embodiment, an ibandronate-containing formulation has a pH of about 2.5, or 2.6, or 2.7, or 2.8, or 2.9, or 3.0, or 3.1, or 3.2, or 3.3, or 3.4, or 3.5, or 3.6, or 3.7, or 3.8, or 3.9, or 4.0, or 4.1, or 4.2, or 4.3, or 4.4, or 4.5, or 4.6, or 4.7, or 4.8, or 4.9, or 5.0. In another embodiment, an ibandronate-containing formulation has a pH between about 2.5 and 3.5, or between about 2.5 and 2.9. In yet another embodiment, an ibandronate-containing formulation has a pH between about 4.6 and 5.5, or between about 4.6 and 5.0.

In another embodiment, a bisphosphonate formulation comprises ibandronate or a physiologically or pharmaceutically acceptable salt of ibandronate, such as ibandronate sodium. Ibandronate-containing formulations can be combined with other compounds or agents, such as pharmacological agents.

In another embodiment, the invention concerns well-tolerated injection solutions that are stable when stored in primary packaging made of glass and contain at least one diphosphonic acid or at least one physiologically acceptable salt of such an acid, processes for producing these solutions and the use of polyethylene glycols to stabilize these solutions.

Bisphosphonate formulations can be of interest for the treatment of hypercalcaemia and can be used as active substances in therapeutic agents for the treatment of osteoporosis and in tumor osteolysis. Bisphosphonates can have preferable efficacy in reducing or inhibiting bone-reabsorption, which is increased in an unnatural manner in many bone diseases, such as, e.g., Morbus Paget, bone tumors, bone metastases, osteoporosis and hyperparathyroidism.

In an embodiment, at room temperature, diphosphonic acids and salts provided herein can be stable for at least 1 year, or at least 2 years, or at least 3 years, or at least 4 years, or at least 5 years, or at least 10 years. An injection solution having at least one diphosphonic acid or at least one acceptable salt or ester of such an acid is stable in a storage container (e.g., glass storage container) when the injection solution has a pH value between about 3.0 and 4.5 and/or contain polyethylene glycols and optionally filled into a container (or injection vessel) that is surface treated to reduce or eliminate reaction with one or more surfaces of the storage container.

Bisphosphonate formulations can be combined or modified with various components, including, without limitation, glidants, lubricants, antioxidants, antimicrobial agents, enzyme inhibitors, stabilizers (including pH stabilizers), retarding agents, preservatives and modifiers. For example, bisphosphonate formulations can be combined or modified with components described in U.S. Patent Publication No. 2009/0214645 to Kramer et al., which is entirely incorporated herein by reference.

In an embodiment, a bisphosphonate formulation, such as an ibandronate-containing formulation, can be combined with one or more of a pH modifier, stabilizer and retarding agent. The pH modifier can be selected from inorganic acids or water-soluble inorganic acids that are solid at ambient temperature, such as, for example sulfamic acid. In an embodiment, pH modifiers can be selected from citric acid, fumaric acid, succininc acid, adipic acid or maleic acid. In another embodiment, pH modifiers can be selected from fumaric acid.

In an embodiment, bisphosphonate formulations can include ethylene glycol. In another embodiment, bisphosphonate formulations can include polyethylene glycol. In yet another embodiment, bisphosphonate formulations can include one or more polymers selected to modify or stabilize the pH of the formulation.

In an embodiment, the addition of polyethylene glycols to a solution having a diphosphonic acid can have the effect of lowering the pH of the solution. Polyethylene glycols having molecular weights between about 100 and 1500 g/mol, or between about 200 and 600 g/mol, can be used. The amount of added polyethylene glycols can be up to about 20 volume percent. In an embodiment, between about 1% and 20%, or between about 2% and 15%, or between about 5% and 10% of polyethylene glycol can be used.

In another embodiment, the solution of the bisphosphonate solution can be reduced in addition to providing polyethylene glycol to the solution. In an embodiment, the pH of a solution having a bisphosphonate and polyethylene glycol is maintained between about 2.5 and 5.5, or between about 3.0 and 4.5.

Bisphosphonate formulations, including ibandronate-containing formulations, can include other pharmaceutically active or inactive ingredients. Such ingredients can be added to provide a desirable fluid property of the formulation, such as a desirable viscosity for administering the formulation using a microinjection device. In an embodiment, a bisphosphonate formulation can include one or more excipients (inactive ingredients), such as dyes, flavors, binders, emollients, fillers, lubricants and preservatives. In another embodiment, an ibandronate-containing formulation can include one or more excipients, such as dyes, flavors, binders, emollients, fillers, lubricants and preservatives. In another embodiment, a bisphosphonate formulation can include one or more of cornstarch, lactose, talc, magnesium stearate, sucrose, gelatin, calcium stearate, silicon dioxide, shellac and glaze. In another embodiment, an ibandronate-containing formulation can include one or more of cornstarch, lactose, talc, magnesium stearate, sucrose, gelatin, calcium stearate, silicon dioxide, shellac, glaze and microcrystalline cellulose. In another embodiment, a bisphosphonate formulation can include one or more of lactose, microcrystalline cellulose, pregelatinized starch, hypromellose, magnesium stearate, titanium dioxide, triacetin, and iron oxide yellow. In another embodiment, an ibandronate formulation can include one or more of lactose, microcrystalline cellulose, pregelatinized starch, hypromellose, magnesium stearate, titanium dioxide, triacetin, and iron oxide yellow. In another embodiment, a bisphosphonate formulation can include one or more of aspartame, gelatin, mannitol, methylparaben sodium and propylparaben sodium. In another embodiment, a bisphosphonate formulation can include one or more of citric acid anhydrous, purified water, sodium benzoate, sodium citrate and sorbitol. In another embodiment, an ibandronate formulation can include one or more of aspartame, gelatin, mannitol, methylparaben sodium and propylparaben sodium. In another embodiment, an ibandronate formulation can include one or more of citric acid anhydrous, purified water, sodium benzoate, sodium citrate and sorbitol.

In an embodiment, a bisphosphonate or ibandronate-containing formulation can include one or more excipients selected from lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup and methyl cellulose. A bisphosphonate or ibandronate-containing formulation can also include one or more of lubricating agents (such as talc); magnesium stearate; mineral oil; wetting agents; emulsifying and suspending agents; preserving agents, such as methyl and propylhydroxy-benzoates; sweetening agents; and flavoring agents. In another embodiment, a bisphosphonate formulation can include one or more of mannitol, edentate disodium dihydrate, trisodium citrate dihydrate and citric acid monohydrate. In another embodiment, a bisphosphonate formulation can include one or more of citric acid anhydrous, hypromellose, lactose, magnesium stearate, cellulose (or microcrystalline cellulose), polyethylene glycol, polysorbate, sodium starch glycolate and titanium dioxide. In another embodiment, a bisphosphonate formulation can include one or more of mannitol, phenol, acetate and water. In another embodiment, a bisphosphonate formulation can include one or more of sodium metabisulfate, benzyl alcohol, sodium hydroxide and water. In another embodiment, an ibandronate formulation can include one or more of mannitol, edentate disodium dihydrate, trisodium citrate dihydrate and citric acid monohydrate. In another embodiment, an ibandronate formulation can include one or more of citric acid anhydrous, hypromellose, lactose, magnesium stearate, cellulose (or microcrystalline cellulose), polyethylene glycol, polysorbate, sodium starch glycolate and titanium dioxide. In another embodiment, an ibandronate formulation can include one or more of mannitol, phenol, acetate and water. In another embodiment, an ibandronate formulation can include one or more of sodium metabisulfate, benzyl alcohol, sodium hydroxide and water.

In an embodiment, a bisphosphonate formulation can include, in addition to the bisphosphonate, one or more of glacial acetic acid, sodium acetate, mannitol, metacresol, hydrochloric acid, sodium hydroxide and water. In another embodiment, a bisphosphonate formulation can include, in addition to the bisphosphonate, one or more of methanol, ethanol, iso-propanol, sodium citrate, hydrochloric acid, ethylene glycol, polyethylene glycol, glycine buffer, maleate, glycerol and ammonium sulfate.

In an embodiment, an ibandronate-containing formulation can include, in addition to ibandronate or derivative of ibandronate, one or more of glacial acetic acid, sodium acetate, mannitol, metacresol, hydrochloric acid, sodium hydroxide and water. In another embodiment, an ibandronate-containing formulation can include, in addition to ibandronate or derivative of ibandronate, one or more of methanol, ethanol, iso-propanol, sodium citrate, hydrochloric acid, ethylene glycol, polyethylene glycol, glycine buffer, maleate, glycerol and ammonium sulfate.

In an embodiment, an bisphosphonate formulation can include, in addition to bisphosphonate, one or more of sodium chloride, glacial acetic acid, sodium acetate and water. In another embodiment, an ibandronate-containing formulation can include, in addition to ibandronate or derivative of ibandronate, one or more of sodium chloride, glacial acetic acid, sodium acetate and water.

Bisphosphonate formulations can be combined or modified with other bisphosphonate formulations, such as, for example, compounds or formulations provided by U.S. Pat. No. 5,662,918 to Winter et al. (“Winter”), entitled “PHARMACEUTICAL AGENTS CONTAINING DIPHOSPHONIC ACIDS AND SALTS THEREOF”, which is entirely incorporated herein by reference. Winter teaches pharmaceutical preparations that are stable upon storage, which contain at least one diphosphonic acid and/or at least one physiologically acceptable salt of such an acid as the active substance. Formulations provided herein can also be combined or modified with other pharmaceutical agents, such as those described in EP 0 170 228, EP 0 197 478, EP 0 224 751, EP 0 252 504, EP 0 252 505, EP 0 258 618, EP 0 350 002, EP 0 273 190, WO 90/00798 and DE 3 500 670, which are entirely incorporated herein by reference.

### Injection and Microinjection Systems

In another aspect of the invention, injection systems are provided for the delivery bisphosphonate compounds provided herein. In embodiments, injection systems include microinjection systems. Microinjection systems provided herein can be configured for subcutaneous, transdermal or intradermal drug delivery. Microinjection systems can provide improved delivery efficiency and absorption times in relation to traditional syringes. A microinjection system can include one or more microneedles configured to deliver bisphosphonate drug formulations, such as, for example, formulations having ibandronate or a salt of ibandronate.

In another embodiment, a microinjection system can include a solid microneedle system having one or more solid microneedles, wherein at least a portion of the one or more solid microneedles are coated with a bisphosphonate drug formulation, such as ibandronate sodium. In another embodiment, a microinjection system can include a hollow microneedle system having one or more hollow microneedles. The one or more hollow microneedles can include fluid passages for directing a formulation having a bisphosphonate drug formulation from a reservoir to a subject.

In some embodiments, solid microneedle systems are provided having one or more microneedles (or microneedle assemblies). In an embodiment, the solid microneedle systems can be configured for the delivery bisphosphonate drug formulations, up to and including about 0.5 mg of a bisphosphonate drug formulation. In another embodiment, solid microneedle systems can include between about 300 and 1500 solid microneedles. Each microneedle can have a height between about 250 and 700 μM tall. In yet another embodiment, each microneedle can be coated with a bisphosphonate-containing drug or vaccine, such as a drug formulation including ibandronate sodium. In an embodiment, the tip of each microneedle can be coated with a bisphosphonate drug formulation. A solid microneedle system can be integrated into a user-wearable device. Upon application, the microneedles penetrate stratum corneum for delivery of the bisphosphonate drug formulation. The microneedles can remain in the skin for a desirable or predetermined period of time, such as a length of time selected to permit the delivery of the bisphosphonate-containing drug to a subject. Such time can be between about 30 seconds and 10 minutes. Bisphosphonate drug formulations can be kept in a dry state, which can enhance stability, allowing for room temperature storage of the formulations. Solid microneedle system can be configured for single or multiple uses.

In an embodiment, a microinjection device having one or more hollow microneedles is provided. The one or more hollow microneedles can be configured to deliver bisphosphonates. In another embodiment, microinjection devices can include a plurality of hollow microneedles. In another embodiment, a hollow microneedle system can be configured for the delivery of a bisphosphonate drug formulation in liquid form, from about 0.01 milliliters (“mL”) up to and including about 3 mL of a bisphosphonate drug formulation, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94; 2.95, 2.96, 2.97, 2.98, 2.99, or 3.00 mL. In another embodiment, a hollow microneedle system can be configured for the delivery of a bisphosphonate drug formulation in liquid form, from about 0.01 ml to 6 mL, or 0.01 mL to 3 mL, or 0.02 mL to 2 mL of a bisphosphonate drug formulation. In another embodiment, hollow microneedle systems can include about 18 hollow microneedles per cm². Each microneedle can have a height of about 900 μm. A hollow microneedle system can be integrated into user-wearable device. Upon application, the microneedles penetrate the skin. Small channels in each microneedle allow for the flow of a fluid having a bisphosphonate drug formulation from the device into the skin. In another embodiment, the delivery time can be between about 10 seconds and 1 hour, or between about 30 seconds and 40 minutes, or between about 1 minute and 30 minutes. The infusion time can be dependent on the viscosity and volume of the bisphosphonate-containing fluid.

In another embodiment, a hollow microneedle system can be configured for the delivery of a formulation comprising ibandronate, ibandronate sodium, ibandronate monosodium salt monohydrate, or other pharmaceutically acceptable salt of ibandronate, from about 0.01 mL up to and including about 3 mL of the formulation, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.99, or 3.00 mL. In another embodiment, a hollow microneedle system can be configured for the delivery of an ibandronate-containing formulation in liquid form, from about 0.01 ml to 6 mL, or 0.01 mL to 3 mL, or 0.02 mL to 2 mL of an ibandronate-containing drug formulation. In an embodiment, hollow microneedle systems can include about 18 hollow microneedles per cm². Each microneedle can have a height of about 900 μm. A hollow microneedle system can be integrated into user-wearable device. Upon application, the microneedles penetrate the skin. Small channels in each microneedle allow for the flow of a fluid having an ibandronate-containing drug formulation from the device into the skin. In another embodiment, the delivery time can be between about 10 seconds and 1 hour, or between about 30 seconds and 40 minutes, or between about 1 minute and 30 minutes. The infusion time can be dependent on the viscosity and volume of the bisphosphonate-containing fluid.

In an embodiment, a microinjection device comprises a plurality of hollow microneedles configured to deliver a bisphosphonate formulation to a subject. In an embodiment, each microneedle is formed of a polymeric material. In another embodiment, each microneedle is formed of a metallic material, such as an elemental metal or a metal alloy. In yet another embodiment, each microneedle is formed of a semiconductor material. In still another embodiment, each microneedle is formed of an insulating material. In still another embodiment, each microneedle is formed of one or more of a metallic material, a semiconductor material and an insulating material.

In an embodiment, a system for subcutaneous, transdermal or intradermal delivery of a bisphosphonate to a subject comprises a bisphosphonate formulation; a microneedle array for delivering the bisphosphonate formulation to a subject; and one or more chambers in fluid communication with the microneedle array, the one or more chambers configured to hold the bisphosphonate formulation. The system can be configured to deliver to a subject a formulation comprising ibandronate, ibandronate sodium, ibandronate monosodium salt monohydrate, or other pharmaceutically acceptable salt of ibandronate.

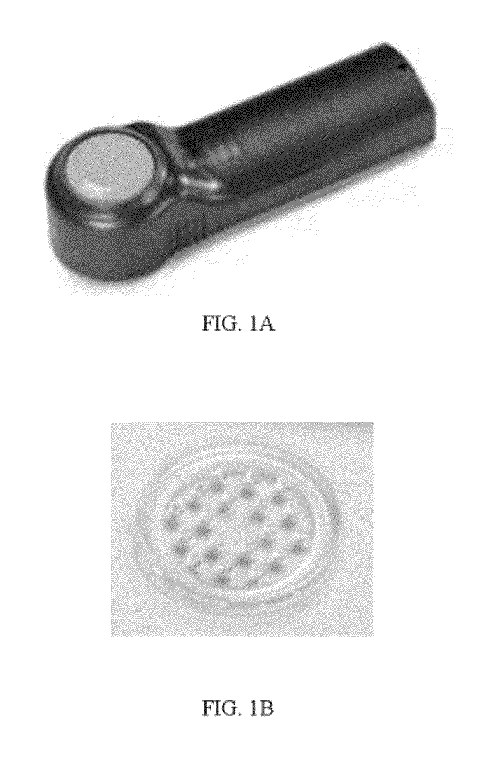

FIG. 1A illustrates a microinjection device configured to deliver bisphosphonates, in accordance with an embodiment of the invention. The microinjection device includes a handle portion configured to permit a user to hold or grip the microinjection device. FIG. 1B illustrates an array of microneedles mountable to the microinjection device of FIG. 1A, in accordance with an embodiment of the invention. Upon application, the microneedles can penetrate the skin of a subject with minimal discomfort. Small channels in each microneedle can allow for fluid flow from the device into the subject's skin.

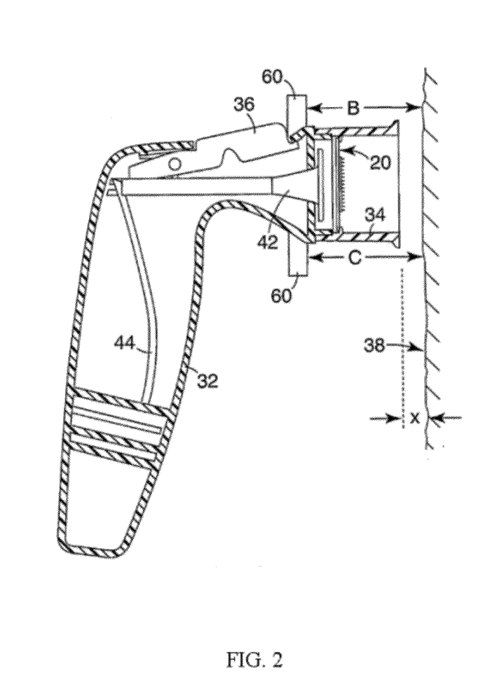

FIG. 2 illustrates a microinjection device having an array of microneedles (also “microneedle array application device” herein), in accordance with an embodiment of the invention. The application device includes a patch 20, microneedle array 22, collar 34, actuator 36, piston 42, driver 44, holding tabs 50 and distance sensors 60. In the illustrated embodiment, the actuator 36 of the device has not been engaged. The driver 44 has stored energy and the piston 42 is not in contact with the patch 20, which is retained within the collar 34 of the application device. The application device has distance sensors 60 that sense distances “B” and “C” between the sensor and a skin surface 38. A user can bring the applicator in proximity to the skin surface 38 so that the distances “B” and “C” 40 can be adjusted so that a distance, “x”, between the end of the collar 34 and the skin surface 38 can be as desired. Once the distance and orientation of the application device is as desired (e.g., when “B” and “C” are equivalent and the distance “x” is less than the distance that the piston 42 protrudes from the device after activation), then the application device can be triggered.

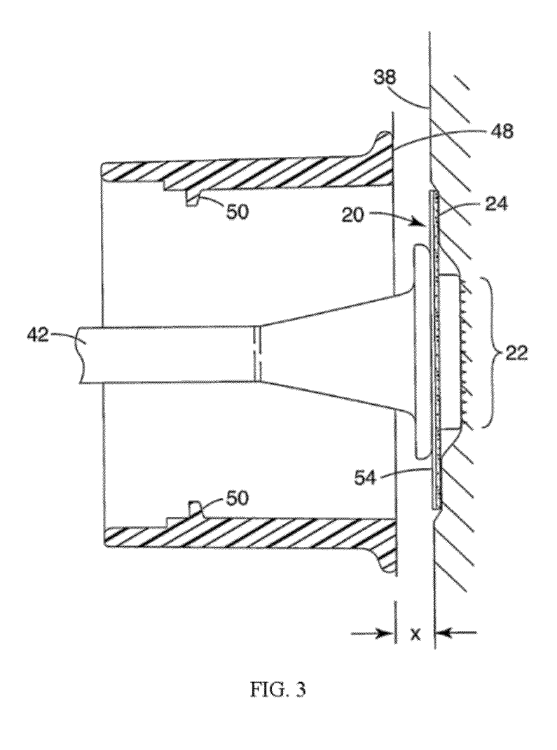

FIG. 3 illustrates a portion of the application device of FIG. 2, in accordance with an embodiment of the invention. The application device is in the second released or triggered position, where the actuator 36 has been engaged, allowing the driver 44 to move the piston 42 towards the patch 20, thereby removing the patch from the holding tabs 50, propelling the patch 20 beyond an open distal end 48 of the collar 34 and pressing the microneedle array 22 and a skin facing adhesive 24 against the skin 38. The piston 42 can then be removed from contact with the patch 20, thereby leaving the patch 20 in place on the skin 38. In an alternative embodiment, the piston 42 can propel the patch 20 and array 22 from the application device and the patch 20 and array 22 can travel part of the distance in air (not shown) before impacting with the skin surface 38.

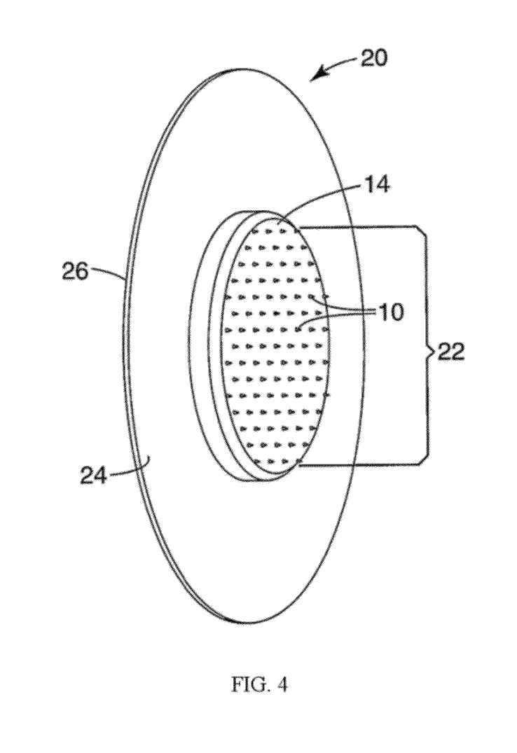

FIG. 4 illustrates a microneedle device comprising a patch 20 in the form of a combination of an array 22, pressure sensitive adhesive 24 and backing 26, in accordance with an embodiment of the invention. A portion of the array 22 is illustrated with microneedles 10 protruding from a microneedle substrate surface 14. The microneedles 10 can be arranged in any desired pattern or distributed over the microneedle substrate surface 14 randomly. In an embodiment, the microneedles 10 are configured for delivering an ibandronate-containing formulation, such as an ibandronate sodium-containing formulation, to a subject. As shown, the microneedles 10 are arranged in uniformly spaced rows. In an embodiment, arrays of the present invention have a skin-facing surface area of more than about 0.1 cm² and less than about 20 cm², or more than about 0.5 cm² and less than about 5 cm². As shown, a portion of the substrate surface 16 of the patch 20 is non-patterned. In an embodiment the non-patterned surface has an area of more than about 1 percent and less than about 75 percent of the total area of the device surface that faces a skin surface of a subject. In an embodiment, the non-patterned surface can have an area of more than about 0.10 square inch (0.65 cm²) to less than about 1 square inch (6.5 cm²). In another embodiment (not shown), the microneedles can be disposed over substantially the entire surface area of the array 22.

The microneedle devices useful in various embodiments of the invention can comprise any of a variety of configurations, such as the structures and configuration disclosed in U.S. Patent Publication No. 2003/0045837 to Delmore et al., U.S. Patent Publication No. 2005/0261631 to Graham et al., U.S. Pat. No. 6,091,975 to Daddona et al., U.S. Pat. No. 6,312,612 to Sherman et al., U.S. Pat. No. 6,379,324 to Garstein et al., and WO/2000/74766 to Garstein et al., which are entirely incorporated herein by reference.

In an embodiment, a microinjection device is provided having an array of microneedles, wherein the microneedles in the array include tapered structures that include at least one channel formed in the outside surface of each microneedle. The microneedles can include bases that are elongated in one direction. The channels in microneedles with elongated bases can extend from one of the ends of the elongated bases towards the tips of the microneedles. The channels formed along the sides of the microneedles can optionally be terminated short of the tips of the microneedles. The microneedle arrays can also include conduit structures formed on the surface of the substrate on which the microneedle array is located. The channels in the microneedles can be in fluid communication with the conduit structures. In another embodiment, each of the microneedles can include a truncated tapered shape and a controlled aspect ratio. In yet another embodiment, microneedles can include blade-like microprotrusions for piercing the skin. In still another embodiment, each of the microneedles can include a hollow central channel. In still another embodiment, each of the microneedles can be hollow and include at least one longitudinal blade at the top surface of tip of a microneedle.

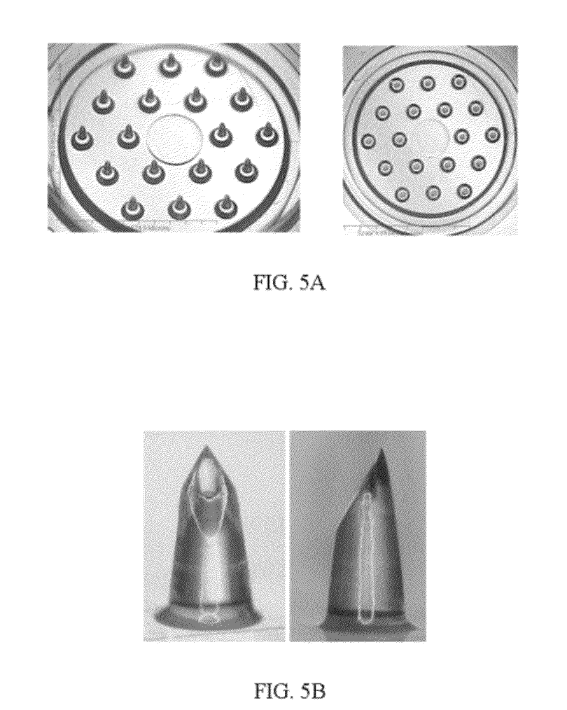

With reference to FIG. 5A, an array of microneedles is shown, in accordance with an embodiment of the invention. FIG. 5B shows a cross-section of a microneedle in the array, in accordance with an embodiment of the invention. The microneedles can be formed of a polymeric material, such as a medical-grade polymeric material. They can be configured to overcome the barrier properties of the stratum corneum to deliver to a subject a bisphosphonate, such as ibandronate sodium. In an embodiment, microneedles can be modeled as mini hypodermic needles, each having a height between about 100 μm and 1000 μm, or between about 300 μm and 950 μm, or between about 500 μm and 900 μm. In an embodiment, the array can include 18 microneedles in an array area of about 1 cm². Each microneedle can include a fluid passage for delivering bisphosphonates, each fluid passage running the length of a microneedle. Each microneedle can include a tip portion configured to pierce a subject's skin.

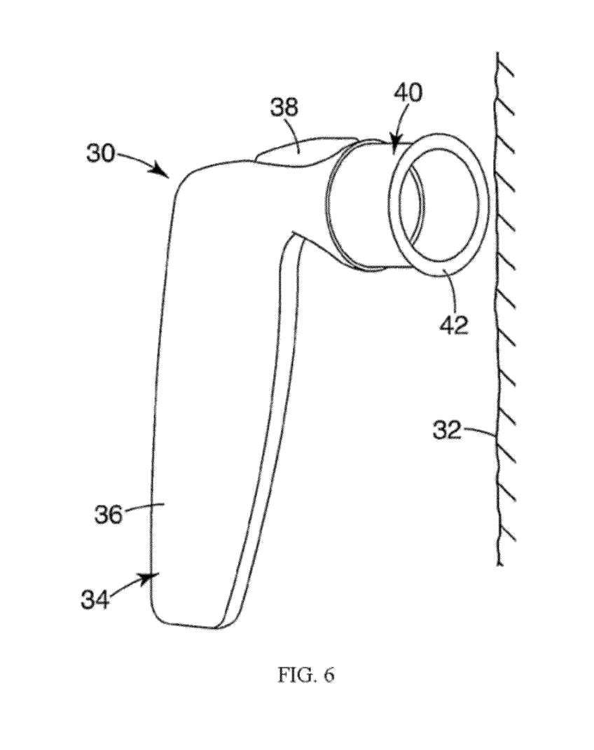

FIG. 6 illustrates a microneedle application device 30 and a skin surface 32, in accordance with an embodiment of the invention. The microneedle device 30 can be used to deliver bisphosphonate compounds, such as ibandronate or a salt of ibandronate (e.g., ibandronate sodium). In an embodiment, the microneedle device 30 can be used to deliver an ibandronate sodium-containing formulation, including pharmacological agents.

With continued reference to FIG. 6, the microneedle application device 30 can be used to deploy patches that include a microneedle array to a surface, such as to the skin surface 32. The device 30 includes a housing 34 with a gripping portion 36, a trigger 38 and a collar 40. The collar 40 defines an outward-facing contact portion 42. In an embodiment, the collar 40 is detachable from the housing 34, and can be disposable or reusable. As shown in FIG. 6, the collar 40 is a unitary member of generally cylindrical shape, and contact portion 42 is generally annular in shape. In further embodiments, the collar 40 can have nearly any shape and configuration. For example, the collar 40 can have a rectangular, triangular, oval, or other shape or combination of shapes. The contact portion 42 will typically have a shape corresponding to the shape of the collar 40. In addition, the collar 40 need not be unitary, and can be configured to form a number of discrete feet or supports that collectively define the contact portion 42.

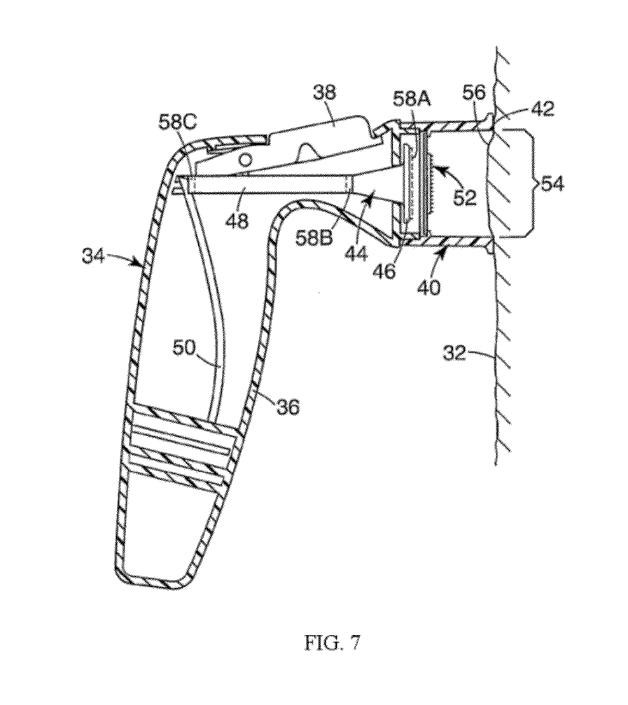

FIG. 7 is a cross sectional side view of the microneedle application device 30 of FIG. 6, in accordance with an embodiment of the invention. The device 30 includes a microneedle array patch 52; the device 30 is positioned against the skin surface 32. The device 30 includes a support member or actuator. In the illustrated embodiment of FIG. 7, the support member or actuator can be a piston 44 having a pad 46 and a shaft 48. In alternative embodiments, any type of mechanical, electromechanical, pneumatic, or other type of support member or actuator can be used.

With continued reference to FIG. 7, a driver 50 capable of storing energy engages the shaft 48 of the piston 44, and can accelerate the piston 44 to a desired velocity. For example, the driver 50 can be in the form of a mechanical spring (e.g., a coil spring, leaf spring, etc.), compressed resilient member (e.g., rubber, etc.), compressed fluids (e.g., air, liquids, etc.), piezoelectric structure, electromagnetic structure, etc. The collar 40 can hold a patch 52, carrying a microneedle array, prior to patch application.

With continued reference to FIG. 7, during operation, the microneedle application device 30 can be positioned with the collar 40 near a desired application site. The contact portion 42 of the collar 40 is placed in contact with the skin surface 32, and the contact portion 42 defines a target patch application site 54 on the skin surface 32. A user can apply force to the microneedle application device 30 at the gripping portion 36 of the housing 34. At least a portion of that force can be transmitted through the collar 40 to the skin 32. That force can be referred to as a “pushdown force”. A “dome” 56 is generally created at the target site 54, as the skin 32 responds to the pushdown force. This “dome” has parameters of height and firmness. Both of these parameters of the dome can be dependent upon the force applied to the applicator during microneedle application device 30 positioning. The depth of penetration of a microneedle array is related to the application site, i.e., soft and fatty areas of a body versus firm muscular areas of the body. Skin characteristics can vary from one individual to another, and particular characteristics of skin can vary across subjects (e.g., patients) and across selected application sites on individual subjects. Such variations can affect characteristics of the dome 56. In addition, a “pushback force” is exerted by the skin 32 in response to the pushdown force. The pushback force is generally directed in a direction directly opposed to the direction of the pushdown force, although specific relationships can be complex and will vary depending on the particular application site.

With continued reference to FIG. 7, a force sensor can be coupled to the piston 44 at either end or anywhere along the length of piston 44, for example, at location 58A, 58B and/or 58C (jointly referred to as sensor 58). The sensor 58 can be capable of sensing applied mechanical forces, such as pushback force at the piston 44. The sensor 58 can be a strain gauge, variable capacitance sensor, or variable resistance sensor. In an embodiment, the sensor 58 can comprise a variable resistance member having a semi-conducting polymer disposed between conductive layers or grids, where the resistance of the variable resistance member varies according to applied force. The variable resistance member can be further configured in a voltage divider, which converts the resistance of the member into a voltage signal output that can be measured to detect force applied to the sensor 58. An example of such a variable resistance member is disclosed in U.S. Pat. No. 5,209,967, which is entirely incorporated herein by reference. Other examples of aspects of such a variable resistance member are disclosed in U.S. Pat. Nos. 5,904,978 and 5,573,626, which are entirely incorporated herein by reference.

With continued reference to FIG. 7, in the microneedle application device 30, the piston 44 is moveable between a stored position and an extended position. In the stored position, energy is stored in the driver 50, and an actuator 38 secures the piston 44 in its stored position. The actuator 38 allows an operator to trigger the release of energy stored in the driver 50 to accelerate the piston 44 through the collar 40 and toward the patch 52.

In an embodiment, the microneedle application device 30 can be used to deliver the microneedle array patch 52 to the skin surface 32, in order to pierce the stratum corneum at the target application site 54 on a subject's skin. For example, the patch application device can be used to deliver bisphosphonates (including any additional pharmacological agent or agents) through the skin in a variation on transdermal delivery, or to the skin for intradermal or topical treatment, such as, e.g., vaccination. Alternatively, the microneedle array patch 52 can be used to pierce the stratum corneum before or after a pharmacological agent is applied to the skin surface in a separate step, thus being used as a pre- or post-treatment step.

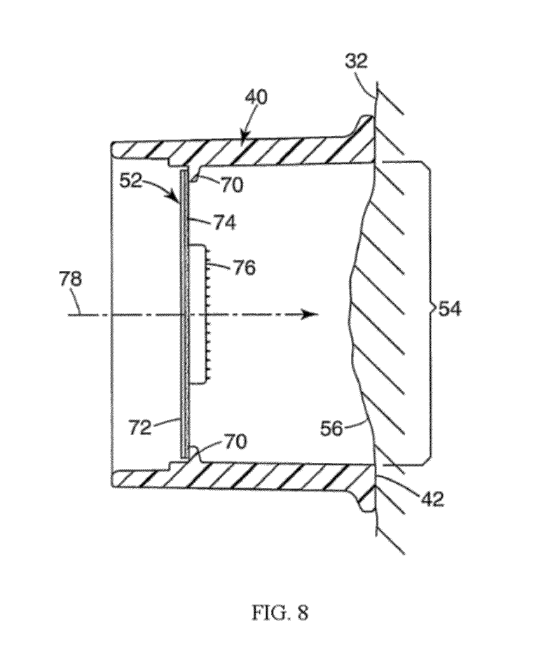

FIG. 8 shows an enlarged cross sectional view of the collar 40 of the microneedle application device 30 of FIGS. 6 and 7, positioned against the skin surface 32, in accordance with an embodiment of the invention. The collar 40 includes obstructions 70 on an interior portion thereof. The obstructions 70 can be configured to retain patches, such as the patch 52. Patch 52 can include a backing 72, an adhesive 74 (e.g., a pressure sensitive adhesive), and a microneedle array 76. A desired patch application path 78 is defined through the collar 40. The path 78 is substantially perpendicular to a plane in which the microneedle array 76 is retained by the obstructions 70 within the collar 40, and is generally perpendicular to the target application site 54. In an embodiment, it is desired that the patch 52 contact the target application site 54 with the patch 52 as close to parallel with the skin surface 32 as possible in order to promote proper microneedle array deployment and proper microneedle penetration of the stratum corneum.

With continued reference to FIG. 8, in operation, the patch 52 is moved along the patch application path 78. This patch movement can be accomplished by mechanically pushing the patch 52 with the piston 44. In alternative embodiments, the microneedle application device 30 can use other means for moving the patch 52. For example, the patch 52 can be moved pneumatically, without contacting a piston.

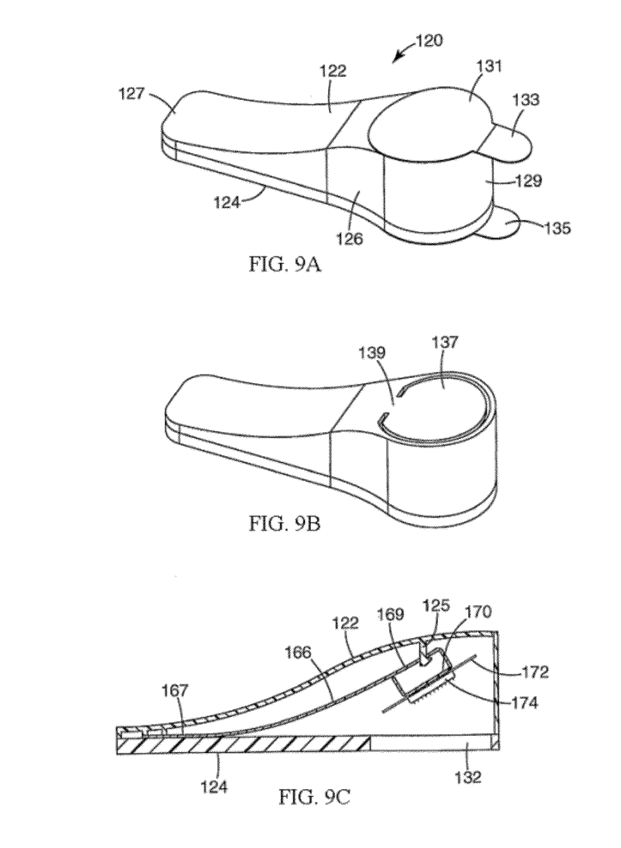

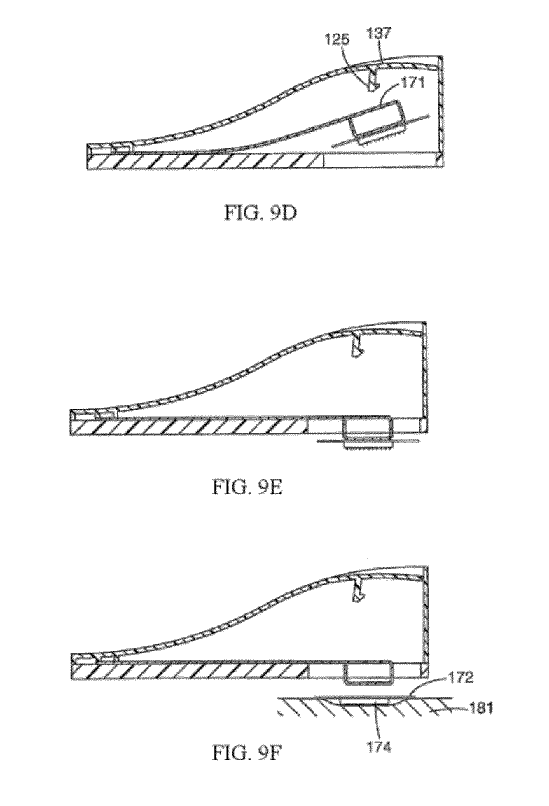

FIG. 9A is a perspective view of an applicator device 120 having a housing 122 that includes a base 124 and an upper cover structure 126, in accordance with an embodiment of the invention. The device is elongate in shape and has a first, tapered end 127 and a second end 129. The second end 129 has a top and bottom sealed by a top peelable seal 131 having a tab 133 and a bottom peelable seal 132 having a tab 135 (only tab 135 is visible in FIG. 9A). FIG. 9B shows the applicator device after the peelable seals 131, 132 have been removed. A trigger 137 is integrally formed in the top surface of the housing 122. The trigger is connected to the top surface of the housing at a single attachment point 139, thus allowing the trigger to be deflected downward by thumb or finger pressure (see FIG. 9D).

FIG. 9C is a cross-sectional view of the device of FIG. 9A, showing a patch 172 mounted on an impactor 170, in accordance with an embodiment of the invention. The impactor 170 is integrally formed with a drive member 166 having a length extending from a fixed end 167 attached to the housing 122 to a movable end 169. The drive member 166 is bendable along its length. A holding mechanism in the form of a latch uses a hook 125 attached to the housing 122. The hook 125 engages with a slot 171 in the movable end 169 of the drive member 166 to hold the movable end 169 of the drive member 166 away from the skin-contacting face 124 of the housing 122. The drive member can be any elongate, bendable member, such as, for example, a leaf spring. In use the device as shown in FIG. 9C is placed against a target surface, such as a skin surface (not shown). Depression of the trigger 137, as shown in FIG. 9D, causes the hook 125 to pivot, thus releasing the movable end 169 of the drive member 166 and allowing the drive member 166 to bias the patch 172 towards the skin-contacting face 124. FIG. 9E shows the drive member 166 fully deployed, having propelled the patch 172 past the skin-contacting face 124 so that the patch is pressed against the skin surface (not shown). FIG. 9F shows the device 120 being removed from the skin surface 181, leaving a patch 172 with a microneedle array 174 in place on the skin surface 181. As shown, the impactor 170 is shown as a curled end of a leaf spring, as this allows for a convenient means for providing a holding mechanism (via the slot 171 in the movable end 169 of the leaf spring) while also providing a separate patch contacting and holding surface. However, any variety of suitable shapes can be used for the movable end 169 of the drive member 166, including a flat leaf spring having no curled end.

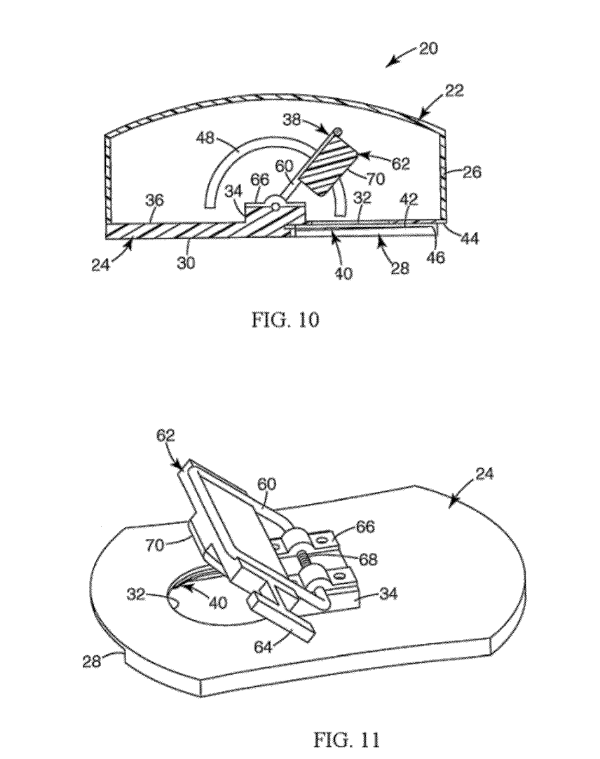

FIG. 10 illustrates an applicator device 20 having a housing 22 that includes a base 24 and an upper cover structure 26, in accordance with an embodiment of the invention. In an embodiment, the applicator device 20 is configured to deliver a bisphosphonate formulation to a subject. In an embodiment, the applicator device 20 is configured to deliver an ibandronate sodium-containing formulation to a subject. The base 24 can be rectangular in shape, and include a recess 28 located on a bottom face 30 thereof. A generally circular opening 32 is defined in the recess 28 of the base 24. A raised portion 34 is formed on an upper face 36 of the base 24 for holding a patch accelerating or patch applicator assembly 38. A mounting structure or retaining portion of the applicator device 20 is formed by a pair of retainers 40, also referred to as a first retainer and a second retainer, connected to the base 24 (only one retainer 40 is visible in FIG. 10). The retainer members 40 are generally elongate and each have a substantially flat upper surface 42 that is generally parallel to and facing a bottom portion 44 of the recess 28, and is spaced from the bottom face 30 (i.e., the skin-contacting face) of the base 24. The pair of retainer members 40 are located on opposite sides of the opening 32 and are connected to the base 24 at one side of the recess 28. The retainer members 40 define an opening 46 at one end for accepting patches between the retainer members 40 and the bottom portion 44 of the recess 28. The upper surfaces 42 of the retainer members 40 can be non-stick or release surfaces. A non-stick or release surface can be achieved, for example, by a non-stick or release coating applied to the upper surfaces 42. The non-stick or release coating can be selected according to the desired use of the applicator device 20. For instance, a release coating, such as a low surface energy silicone, fluoropolymer, or fluoro-silicone release coating, can be selected based upon the adhesives used with patches applied using the patch application device 20. In further embodiments, a blade or other cutting means can be provided as part of the mounting structure, for separating portions of items from patches mounted on the applicator.

With continued reference to FIG. 10, the upper cover structure 26 is connected to the base 24 at or near a perimeter of the base 24. The upper cover structure 26 is shaped to fit on the base 24, and defines a volume, which is selected to provide space for the patch accelerating assembly 38. In some embodiments, the housing 22 can also provide space for storing patches (e.g., a roll of patches) for eventual deployment by the applicator device 20. A slot 48 is defined in a side portion of the upper cover structure 26. In the illustrated embodiment of FIG. 10, the slot 48 is arcuate in shape and generally resembles a half circle, with the open portion of the half circle facing the base 24 of the housing 22. Both the base 24 and the upper cover structure 26 can be formed of a polymeric material.

FIG. 11 is a perspective view of a portion of the applicator device 20 of FIG. 10 with the upper cover portion 26 omitted to show interior portions of the device 20. As shown in FIG. 11, the patch acceleration assembly 38 includes a frame member 60, an impactor 62, a handle 64, a bracket 66, and a torsion spring 68. The torsion spring 68 serves as a drive member to bias the impactor relative to the housing. The bracket 66 is mounted to the raised portion 34 of the base 24 of the housing 22 and pivotally retains the frame member 60. In some instances the bracket 66 can be directly affixed to the base 24, for example, if the base has sufficient thickness to allow for placement of the torsion spring 68. The frame member 60 can be a wire formed as a rectangular loop. The impactor 62 is attached to the frame member 60 opposite the bracket 66, and is the portion of the patch acceleration assembly 38 that interfaces with a patch to move it (i.e., to accelerate it), that is, it is the patch contacting portion of the device. The impactor 62 has a patch contacting surface 70 that is configured according to characteristics of a desired application, for instance, based upon the shape of a patch to be applied. In the embodiment shown in FIG. 10, the patch contacting surface 70 is configured so that it is generally parallel to and aligned with the frame member 60. Furthermore, it will be generally aligned with the bottom face 30 of the device 20 when fully deployed. It other embodiments, the patch contacting surface 70 can be configured so that it is at another angle with respect to the frame member 60, and with respect to the bottom face 30 of the device 20 when fully deployed. Other such angles are possible. In an embodiment, the patch contacting surface 70 can be aligned so as to form an angle of between 4 and 15 degrees with the plane of the frame member. In an embodiment, the angle of the patch contacting surface 70 can be selected so that it is aligned with the back of the a patch resting on retaining members 40 when the patch contacting surface 70 contacts the patch. The impactor 62 can be formed of a polymer material. The handle 64 extends from the impactor 62, and can be integrally formed with the impactor 62. The handle 64 is arranged to protrude through the slot 48 in the upper cover structure 26 of the housing 22, allowing the impactor 62 position to be manipulated from outside the housing 22. It should be understood that FIG. 10 represents one configuration for manipulating the patch acceleration assembly 38. For example, a slot can be provided on the upper cover portion 26, thereby allowing the handle 64 or any other suitable actuation protrusion to protrude through the upper cover portion 26. Furthermore, the method for manipulating the patch acceleration assembly 38 need not be by means of a direct mechanical connection. For example, various linkages or gears can be provided such that a button or knob on the exterior of the housing 22 can be pressed or turned to manipulate the patch acceleration assembly 38. In a further example, the patch acceleration assembly 38 can be moved by a motor or solenoid that is electrically controlled by a button or knob on the exterior of the housing 22.

With continued reference to FIG. 11, the torsion spring 68 biases the frame 60 of the patch acceleration assembly 38 relative to the base 24 of the housing 22. The torsion spring 68 can be a conventional coiled spring steel torsion spring. By default, the torsion spring 68 biases the frame 60, and therefore also the impactor 62, toward the opening 32 in the base 24 of the housing 22. In a substantially de-energized state, the impactor is at rest and positioned near the opening 32 in the base 24 of the housing 22. By moving the handle 64 to position the impactor 62 away from the opening, along an arcuate path that can be defined by movement of the handle 64 along the slot 48 in the upper cover structure 26 of the housing 22, an operator can store potential energy in the torsion spring 68. Energy stored in the torsion spring 68 can be used to accelerate the impactor 62 toward a patch and also to accelerate a patch that has contacted the impactor 62. The amount of energy stored in the torsion spring 68 will vary depending on the amount of displacement of the impactor 62 away from the opening 32 and along the arcuate path. The appropriate torsion spring constant will depend upon a number of parameters, including the mass of the patch acceleration assembly, the mass of the patch, the arc length through which the patch acceleration assembly travels, and the desired speed of the patch on impact with a surface. The torsion spring constant can be more than about 0.5 Newton*mm/degree, or more than about 2.0 Newton*mm/degree. The torsion spring constant can be less than about 5.0 Newton*mm/degree, or less than about 4.0 Newton*mm/degree. The impactor 62 can be held at various points along the arcuate path either manually or, in some embodiments, with holding means (not shown) that engage and temporarily secure the handle 64 along the slot 48 in the upper cover structure 26 of the housing 22. In some embodiments, demarcations or other indicators (e.g., a force readout display) can be provided for indicating the levels of force associated with particular degrees of displacement of the impactor 62 along the arcuate path.

The range of angular travel of the patch acceleration assembly will often be less than about 170 degrees and sometimes less than about 110 degrees. The range of angular travel of the patch acceleration assembly will often be more than about 10 degrees and sometimes more than about 60 degrees. The mass of the patch acceleration assembly will often be more than about 1 gram and sometimes more than about 5 grams. The mass of the patch acceleration assembly will often be less than about 100 grams and sometimes less than about 30 grams.

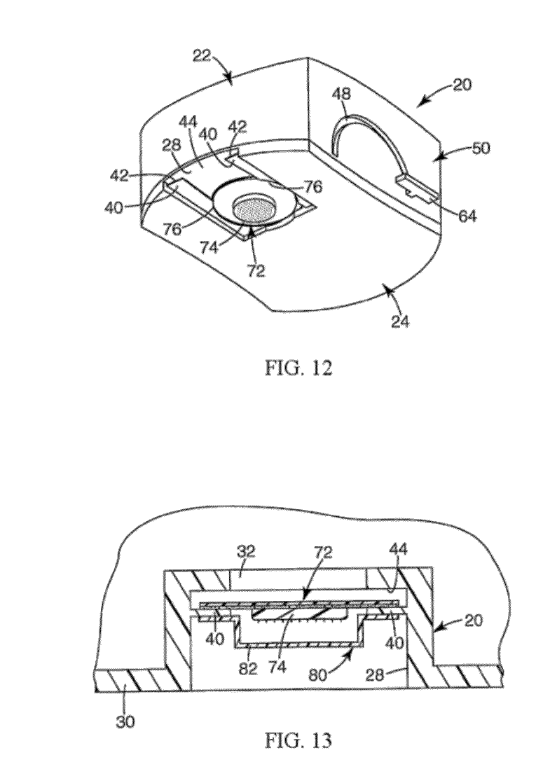

FIG. 12 is a perspective view of a patch 72 (e.g., a patch 72 carrying a microneedle array 74) mounted on the applicator device 20, in accordance with an embodiment of the invention. In an embodiment, the applicator device 20, including the patch 72, is configured to deliver a bisphosphonate formulation to a subject. In an embodiment, applicator device 20 is configured to deliver an ibandronate sodium-containing formulation to a subject. The patch 72 is disposed between the retainer members 40 and the bottom portion 44 of the recess 28 in the base 24 of the housing 22. The microneedle array 74 faces away from the opening 32 in the base 24 of the housing 22. The patch 72, which can have adhesive surrounding the microneedle array 74 on the surface facing away from the patch application device 20, contacts the upper surfaces 42 of the retainer members 40, but is generally not adhered firmly to the retainer members 40 due to the release character of the upper surfaces 42. In a fully mounted position, as shown in FIG. 12, microneedle array carried on the patch 72 is generally aligned relative to the opening 32 in the base 24 of the housing 22 (the opening 32 is not visible in FIG. 12).

With continued reference to FIG. 12, the retainer members 40 have cutaway portions 76 that provide an enlarged, partially circular open region that is generally aligned with the opening 32 on the bottom portion 44 of the recess 28 of the base 24 of the housing 22. The wider, open region defined by the cutaway portions 76 facilitates patch application by reducing the amount of deflection of the patch 72 required during deployment to move the patch 72 from a mounted position on the applicator device 20 to a target location. Such cutaway portions 76 can be omitted if, for example, the patch has a generally rectangular shape.

FIG. 13 is a partial cross-sectional view of a microneedle array cartridge 80, having a patch 72 and a cover 82, mounted on an applicator device 20. In an embodiment, the applicator device 20 is similar to the applicator device of FIG. 12. The microneedle array cartridge 80 includes a microneedle array 74. In an embodiment, the microneedle array 74 is configured to deliver bisphosphonate formulations to a subject. Mounting the patch 72 on the applicator device 20 includes the following steps. The cartridge 80 is partially slid onto the retainer members 40. Then the cartridge 80 is slid further along the retainer members 40, simultaneously separating the cover 82 from the patch 72, until the patch 72 is fully mounted on the applicator device 20 (e.g., such that the microneedle array 74 is aligned with the opening 32 defined in the bottom portion 44 of the recess 28). The cover 82 is removed from (i.e., separated from) the patch 72 to uncover and expose the microneedle array 74 prior to microneedle deployment.

Microinjection methods, devices and systems provided herein can be combined or modified with other injection or microinjection methods, devices and systems, including methods, devices and systems for manufacturing microinjection devices and components (such as, e.g., microneedles). For example, the microneedle devices provided herein can be combined or modified with devices, apparatuses, systems and methods (such as methods of manufacturing) described in U.S. Patent Publication Nos. 2003/0045837 to Delmore et al., 2003/0135161 to Fleming et al., 2005/0143713 to Delmore et al., 2005/0187521 to Fleming et al., 2005/0261631 to Clarke et al., 2006/0195067 to Wolter et al., 2007/0083151 to Carter, 2007/0191761 to Boone et al., 2008/0009811 to Cantor, 2008/0009825 to Ringsred et al., 2008/0039805 to Frederickson et al., 2008/0051699 to Choi et al., 2008/0088066 to Ferguson et al., 2008/0102192 to Johnson et al., 2008/0108958 to Carter et al., 2008/0114298 to Cantor et al., 2008/0195035 to Frederickson et al., 2008/0208146 to Brandwein et al., 2008/0262416 to Duan et al., 2008/0275400 to Ferguson, 2008/0287858 to Duan, 2008/0294116 to Wolter et al., 2008/0319404 to Pekurovsky et al., 2009/0099537 to DeVoe et al., 2009/0171314 to Ferguson, 2009/0198189 to Simons et al., 2009/0277794 to Trice et al., 2010/0159197 to Ferguson et al., 20100193997 to Frederickson et al. and 2010/0222743 to Frederickson et al., which are entirely incorporated herein by reference, and U.S. Pat. Nos. 6,881,203 to Delmore et al. and 6,908,453 to Fleming et al., which are entirely incorporated herein by reference. As another example, the microneedle (or microinjection) devices provided herein can be combined or modified with devices, apparatuses, systems and methods (including methods of manufacturing) described in U.S. Patent Publication No. 2004/0249339, U.S. Patent Publication No. 2005/0154350, U.S. Patent Publication No. 2005/0137536, U.S. Patent Publication No. 2003/0135201, U.S. Patent Publication No. 2009/0043250, U.S. Patent Publication No. 2003/0135158, U.S. Patent Publication No. 2003/0135166, U.S. Patent Publication No. 2003/0135167, U.S. Patent Publication No. 2009/0062752, U.S. Patent Publication No. 2005/0119618, U.S. Patent Publication No. 2006/0030838, U.S. Patent Publication No. 2004/0106904, U.S. Patent Publication No. 2009/0118672, U.S. Patent Publication No. 2009/0240232, U.S. Patent Publication No. 2003/0149397, U.S. Patent Publication No. 2002/0156418, U.S. Patent Publication No. 2002/0151842, U.S. Patent Publication No. 2002/0161329, U.S. Patent Publication No. 2009/0157005, U.S. Patent Publication No. 2009/0198185, U.S. Patent Publication No. 2010/0217191, U.S. Patent Publication No. 2002/0169416, U.S. Patent Publication No. 2006/0189939, U.S. Patent Publication No. 2006/0189939, U.S. Patent Publication No. 2009/0093763, U.S. Pat. No. 6,939,324, U.S. Pat. No. 7,150,409, U.S. Pat. No. 7,481,792, U.S. Pat. No. 7,530,968, U.S. Pat. No. 7,187,969, U.S. Pat. No. 6,616,627, U.S. Pat. No. 6,406,455, U.S. Pat. No. 6,314,317, U.S. Pat. No. 6,960,184, U.S. Pat. No. 6,490,483, U.S. Pat. No. 6,939,324, U.S. Pat. No. 7,027,478, U.S. Pat. No. 6,230,051, WO/2000/035520, WO/2001/051109, WO/2002/051470, WO/2002/050584, WO/2003/024507, WO/2003/026732, WO/2004/033021, and WO/2007/115039, which are entirely incorporated herein by reference.

### Methods and Systems for Treating Subjects

In one aspect of the invention, microinjection devices are used to deliver bisphosphonate formulations (or drug formulations) to subjects. Microinjection devices for delivering bisphosphonate formulations can be selected from any microinjection or microneedle devices provided herein. In an embodiment, a microinjection device having one or more microneedles is used to deliver a bisphosphonate formulation to a subject. In an embodiment, a microinjection device having a plurality of microneedles can be used to deliver an ibandronate sodium-containing formulation to a subject. In an embodiment, the ibandronate sodium-containing formulation is delivered in a transdermal fashion. In another embodiment, the ibandronate sodium-containing formulation is delivered to the subject in an intradermal fashion.

In an embodiment, microinjection devices having bisphosphonate formulations can be used to treat bone disease. In another embodiment, microinjection devices having bisphosphonate formulations can be used to treat osteoporosis. In yet another embodiment, microinjection devices having bisphosphonate formulations can be used to treat osteoarthritis. In still another embodiment, microinjection devices having bisphosphonate formulations can be used to treat osteopenia. In still another embodiment, microinjection devices having bisphosphonate formulations can be used to treat osteonecrosis. In still another embodiment, microinjection devices having bisphosphonate formulations can be used to treat osteomalacia. In still another embodiment, microinjection devices having bisphosphonate formulations can be used to treat osteogenesis imperfecta. In still another embodiment, microinjection devices having bisphosphonate formulations can be used to treat osteosclerosis. In still another embodiment, microinjection devices having bisphosphonate formulations can be used to treat osteomyelitis. In still another embodiment, microinjection devices having bisphosphonate formulations can be used to treat osteopetrosis. In still another embodiment, microinjection devices having bisphosphonate formulations can be used to treat Paget disease. In still another embodiment, microinjection devices having bisphosphonate formulations can be used to treat rickets. In still another embodiment, a microinjection device having microneedles (such as any of the devices described above) having an ibandronate sodium-containing formulation can be used to treat osteoporosis.

In an embodiment, a bisphosphonate formulation is administered to a subject by subcutaneous, transdermal or intradermal administration. In another embodiment, subcutaneous, transdermal or intradermal administration is by drug vehicle interaction. In yet another embodiment, subcutaneous, transdermal or intradermal administration is by the use of ion pairs or coacervates. In still another embodiment, subcutaneous, transdermal or intradermal administration is by vesicles and particles. In still another embodiment, subcutaneous, transdermal or intradermal administration is by liposomes and analogues. In still another embodiment, subcutaneous, transdermal or intradermal administration is with the use of high velocity particles. In still another embodiment, subcutaneous, transdermal or intradermal administration is by removing, bypassing or modifying the stratum corneum. In still another embodiment, subcutaneous, transdermal or intradermal administration is by hydration. In still another embodiment, subcutaneous, transdermal or intradermal administration is with the use of chemical enhances. In still another embodiment, subcutaneous, transdermal or intradermal administration is by microneedle injection. In still another embodiment, subcutaneous, transdermal or intradermal administration is by ablation. In still another embodiment, subcutaneous, transdermal or intradermal administration is by follicular delivery. In still another embodiment, subcutaneous, transdermal or intradermal administration is by electrically assisted methods. In still another embodiment, subcutaneous, transdermal or intradermal administration is by ultrasound. In still another embodiment, subcutaneous, transdermal or intradermal administration is by iontophoresis. In still another embodiment, subcutaneous, transdermal or intradermal administration is by electroporation.

In an embodiment, a bisphosphonate formulation is administered subcutaneously, transdermally or intradermally with the aid of iontophoresis, which can involve non-invasively propelling high concentrations of a charged substance, including a bisphosphonate formulation formulation, transdermally or intradermally by a repulsive electromotive force using a small electrical charge applied to an iontophoretic chamber containing a similarly charged active agent, such as a bisphosphonate formulation, and its vehicle. In another embodiment, an ibandronate-containing formulation is administered transdermally or intradermally with the aid of iontophoresis. In yet another embodiment, a device having one or more chambers filled with a solution containing a bisphosphonate formulation is provided. The bisphosphonate formulation can provided in the one or more chambers with a solvent to aid in (or facilitate) delivery. The device can include one or both of a positively charged chamber for repelling a positively charged chemical and a negatively charged chamber for repelling a negatively charged chemical into the skin of a subject.

In an embodiment, a bisphosphonate formulation is administered subcutaneously, transdermally or intradermally with the aid of ultrasound or ultrasonic energy (also “ultrasound” herein). In another embodiment, an ibandronate-containing formulation is administered transdermally or intradermally with the aid of ultrasound. The application of ultrasound to the skin can increase the permeability of skin to a bisphosphonate formulation, which can enable the delivery of a bisphosphonate formulation, such as an ibandronate-containing formulation, through the skin.

In an embodiment, a bisphosphonate formulation is administered subcutaneously, transdermally or intradermally with the aid of electroporation. In another embodiment, an ibandronate-containing formulation is administered transdermally or intradermally with the aid of electroporation. In yet another embodiment, a device is provided for applying an electric field to an area of a subject's body in which transdermal administration of a bisphosphonate formulation is desired, such as, for example, a portion of a subject's arm. The application of the electric field can facilitate the transdermal delivery of the bisphosphonate formulation, such as an ibandronate-containing formulation, to the subject.

In an embodiment, a bisphosphonate formulation is administered subcutaneously, transdermally or intradermally by microneedle injection. In another embodiment, an ibandronate-containing formulation is administered transdermally or intradermally by microneedle injection. Microneedle injection can include use of a microneedle device, such as a microneedle device of various embodiments of the invention.

In an embodiment, a first user employs a microinjection device having a bisphosphonate formulation to deliver the bisphosphonate formulation to a subject. In an embodiment, the first user is a doctor or healthcare professional and the subject is a patient. In another embodiment, the first user is a caregiver and the second user is a subject under the caregiver's care. In another embodiment, the first user is a friend or relative of the subject.

In another embodiment, a subject employs a microinjection device having a bisphosphonate formulation to self-administer the bisphosphonate formulation.

It will be appreciated that the term “user”, as used herein, can refer to an individual using a microinjection device to administer a bisphosphonate formulation to another individual, such as a subject, or to an individual using the microinjection device to administer the bisphosphonate formulation to her or himself. The term “subject”, as used herein, can refer to any individual under treatment by another individual, such as a healthcare provider (e.g., physician, physician's assistant, nurse) or a care provider, or to an individual administering the bisphosphonate formulation to himself or herself (i.e., self administration). A “subject” includes asymptomatic individuals and symptomatic individuals, such as a patient.

In an embodiment, a bisphosphonate formulation can have bisphosphonate concentration (mg bisphosphonate/mL formulation or solution) of about 0.1 mg/3 mL, or 0.2 mg/3 mL, or 0.3 mg/3 mL, or 0.4 mg/3 mL, or 0.5 mg/3 mL, or 0.6 mg/3 mL, or 0.7 mg/3 mL, or 0.8 mg/3 mL, or 0.9 mg/3 mL, or 1.0 mg/3 mL, or 1.1 mg/3 mL, or 1.2 mg/3 mL, or 1.3 mg/3 mL, or 1.4 mg/3 mL, or 1.5 mg/3 mL, or 1.6 mg/3 mL, or 1.7 mg/3 mL, or 1.8 mg/3 mL, or 1.9 mg/3 mL, or 2.0 mg/3 mL, or 2.1 mg/3 mL, or 2.2 mg/3 mL, or 2.3 mg/3 mL, or 2.4 mg/3 mL, or 2.5 mg/3 mL, or 2.6 mg/3 mL, or 2.7 mg/3 mL, or 2.8 mg/3 mL, or 2.9 mg/3 mL, or 3.0 mg/3 mL, or 3.1 mg/3 mL, or 3.2 mg/3 mL, or 3.3 mg/3 mL, or 3.4 mg/3 mL, or 3.5 mg/3 mL, or 3.6 mg/3 mL, or 3.7 mg/3 mL, or 3.8 mg/3 mL, or 3.9 mg/3 mL, or 4.0 mg/3 mL, or 4.1 mg/3 mL, or 4.2 mg/3 mL, or 4.3 mg/3 mL, or 4.4 mg/3 mL, or 4.5 mg/3 mL, or 4.6 mg/3 mL, or 4.7 mg/3 mL, or 4.8 mg/3 mL, or 4.9 mg/3 mL, or 5.0 mg/3 mL. In another embodiment, a bisphosphonate formulation can have a bisphosphonate concentration between about 0.1 mg/3 mL and 5 mg/3 mL, or between about 1 mg/3 mL and 4 mg/3 mL, or between about 2 mg/3 mL and 3.5 mg/3 mL. In yet another embodiment, bisphosphonate formulation can have a bisphosphonate concentration of about 1 mg/3 mL, or 2 mg/3 mL, or 3 mg/3 mL, or 4 mg/3 mL, or 4 mg/3 mL, or 5 mg/3 mL. In still another embodiment, a bisphosphonate formulation can have a bisphosphonate concentration between about 3.0 mg in 3 mL and 3.5 mg in 3 mL solution, or between about 3.3 mg in 3 mL and 3.4 mg in 3 mL solution. In still another embodiment, a bisphosphonate formulation can have a bisphosphonate concentration of about 3.3 mg in 3 mL solution, or about 3.325 mg in 3 mL solution, or about 3.35 mg in 3 mL solution, or about 3.375 mg in 3 mL solution, or about 3.4 mg in 3 mL solution.

In an embodiment, an ibandronate-containing formulation can have an ibandronate or ibandronate salt (e.g., ibandronate sodium, ibandronate monosodium salt monohydrate ibandronate, or other pharmaceutically acceptable salt of ibandronate) concentration (mg ibandronate/mL formulation or solution) of about 0.1 mg/3 mL, or 0.2 mg/3 mL, or 0.3 mg/3 mL, or 0.4 mg/3 mL, or 0.5 mg/3 mL, or 0.6 mg/3 mL, or 0.7 mg/3 mL, or 0.8 mg/3 mL, or 0.9 mg/3 mL, or 1.0 mg/3 mL, or 1.1 mg/3 mL, or 1.2 mg/3 mL, or 1.3 mg/3 mL, or 1.4 mg/3 mL, or 1.5 mg/3 mL, or 1.6 mg/3 mL, or 1.7 mg/3 mL, or 1.8 mg/3 mL, or 1.9 mg/3 mL, or 2.0 mg/3 mL, or 2.1 mg/3 mL, or 2.2 mg/3 mL, or 2.3 mg/3 mL, or 2.4 mg/3 mL, or 2.5 mg/3 mL, or 2.6 mg/3 mL, or 2.7 mg/3 mL, or 2.8 mg/3 mL, or 2.9 mg/3 mL, or 3.0 mg/3 mL, or 3.1 mg/3 mL, or 3.2 mg/3 mL, or 3.3 mg/3 mL, or 3.4 mg/3 mL, or 3.5 mg/3 mL, or 3.6 mg/3 mL, or 3.7 mg/3 mL, or 3.8 mg/3 mL, or 3.9 mg/3 mL, or 4.0 mg/3 mL, or 4.1 mg/3 mL, or 4.2 mg/3 mL, or 4.3 mg/3 mL, or 4.4 mg/3 mL, or 4.5 mg/3 mL, or 4.6 mg/3 mL, or 4.7 mg/3 mL, or 4.8 mg/3 mL, or 4.9 mg/3 mL, or 5.0 mg/3 mL. In another embodiment, an ibandronate-containing formulation can have an ibandronate or ibandronate salt (e.g., ibandronate sodium) concentration between about 0.1 mg/3 mL and 5 mg/3 mL, or between about 1 mg/3 mL and 4 mg/3 mL, or between about 2 mg/3 mL and 3.5 mg/3 mL. In yet another embodiment, an ibandronate-containing formulation can have an ibandronate sodium concentration of about 1 mg/3 mL, or 2 mg/3 mL, or 3 mg/3 mL, or 4 mg/3 mL, or 4 mg/3 mL, or 5 mg/3 mL. In still another embodiment, an ibandronate-containing formulation can have an ibandronate, ibandronate sodium, ibandronate monosodium salt monohydrate, or other pharmaceutically acceptable salt of ibandronate concentration between about 3.0 mg in 3 mL and 3.5 mg in 3 mL solution, or between about 3.3 mg in 3 mL and 3.4 mg in 3 mL solution. In still another embodiment, an ibandronate-containing formulation can have an ibandronate, ibandronate sodium, ibandronate monosodium salt monohydrate, or other pharmaceutically acceptable salt of ibandronate concentration of about 3.3 mg in 3 mL solution, or about 3.325 mg in 3 mL solution, or about 3.35 mg in 3 mL solution, or about 3.375 mg in 3 mL solution, or about 3.4 mg in 3 mL solution.

Unless the context indicates otherwise, formulation volumes, when used in association with doses (mg), are used to illustrate concentrations and may not necessarily be the volumes of formulations delivered to subjects. In an example, a microinjection device is loaded with about 2 mL of an ibandronate-containing formulation having an ibandronate concentration of about 3.3 mg in 3 mL.

In an embodiment, a microinjection device, such as any device provided herein, is used to deliver a bisphosphonate, including ibandronate, to a subject from once a day to once a month. In another embodiment, a microinjection device, such as any device provided herein, is used to deliver ibandronate to a subject from once a day to once a week. In another embodiment, a microinjection device, such as any device provided herein, is used to deliver a bisphosphonate, including ibandronate, to a subject from once a day to once every other day. In another embodiment, a microinjection device, such as any device provided herein, is used to deliver a bisphosphonate, including ibandronate, to a subject once a day, or twice a day, or three times per day, or four times per day, or five times per day, or six times per day, or seven times per day, or eight times per day, or nine times per day, or ten times per day, or eleven times per day, or twelve times per day, or thirteen times per day, or fourteen times per day, or fifteen times per day, or sixteen times per day, or seventeen times per day, or eighteen times per day, or nineteen times per day, or twenty times per day, or twenty one times per day, or twenty two times per day, or twenty three times per day, or twenty four times per day.

In an embodiment, a microinjection device is used to deliver a bisphosphonate or a bisphosphonate-containing formulation to a subject at a dose (mg bisphosphonate) of about 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg. In another embodiment, a microinjection device is used to deliver ibandronate or an ibandronate formulation to a subject at a dose (mg ibandronate) of about 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg.

In an embodiment, a microinjection device is used to deliver a bisphosphonate or a bisphosphonate-containing formulation to a subject at a dose (mg bisphosphonate) of at least about 0.01 mg, or 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg, or more, and in a volume of the formulation of at least about 0.01 mL, or 0.1 mL, or 1 mL, or 1.5 mL, or 2 mL, or 3 mL, or 4 mL, or 5 mL, or 6 mL, or 7 mL, or 8 mL, or 9 mL, or 10 mL, or more. In another embodiment, a microinjection device is used to deliver ibandronate or an ibandronate formulation to a subject at a dose (mg ibandronate) of at least about 0.01 mg, or 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg, or more, and in a volume of the formulation of at least about 0.01 mL, or 0.1 mL, or 1 mL, or 1.5 mL, or 2 mL, or 3 mL, or 4 mL, or 5 mL, or 6 mL, or 7 mL, or 8 mL, or 9 mL, or 10 mL, or more.

In an embodiment, a microinjection device is used to deliver a bisphosphonate or a bisphosphonate-containing formulation to a subject at a regimen (volume) of at least about 0.1 mL, or 0.2 mL, or 0.3 mL, or 0.4 mL, or 0.5 mL, or 0.6 mL, or 0.7 mL, or 0.8 mL, or 0.9 mL, or 1.0 mL, or 1.1 mL, or 1.2 mL, or 1.3 mL, or 1.4 mL, or 1.5 mL, or 1.6 mL, or 1.7 mL, or 1.8 mL, or 1.9 mL, or 2.0 mL, or 2.1 mL, or 2.2 mL, or 2.3 mL, or 2.4 mL, or 2.5 mL, or 2.6 mL, or 2.7 mL, or 2.8 mL, or 2.9 mL, or 3.0 mL, or 3.1 mL, or 3.2 mL, or 3.3 mL, or 3.4 mL, or 3.5 mL, or 3.6 mL, or 3.7 mL, or 3.8 mL, or 3.9 mL, or 4.0 mL, or 4.1 mL, or 4.2 mL, or 4.3 mL, or 4.4 mL, or 4.5 mL, or 4.6 mL, or 4.7 mL, or 4.8 mL, or 4.9 mL, or 5.0 mL In another embodiment, a microinjection device is used to deliver ibandronate or an ibandronate-containing formulation to a subject at a regimen (volume) of at least about 0.1 mL, or 0.2 mL, or 0.3 mL, or 0.4 mL, or 0.5 mL, or 0.6 mL, or 0.7 mL, or 0.8 mL, or 0.9 mL, or 1.0 mL, or 1.1 mL, or 1.2 mL, or 1.3 mL, or 1.4 mL, or 1.5 mL, or 1.6 mL, or 1.7 mL, or 1.8 mL, or 1.9 mL, or 2.0 mL, or 2.1 mL, or 2.2 mL, or 2.3 mL, or 2.4 mL, or 2.5 mL, or 2.6 mL, or 2.7 mL, or 2.8 mL, or 2.9 mL, or 3.0 mL, or 3.1 mL, or 3.2 mL, or 3.3 mL, or 3.4 mL, or 3.5 mL, or 3.6 mL, or 3.7 mL, or 3.8 mL, or 3.9 mL, or 4.0 mL, or 4.1 mL, or 4.2 mL, or 4.3 mL, or 4.4 mL, or 4.5 mL, or 4.6 mL, or 4.7 mL, or 4.8 mL, or 4.9 mL, or 5.0 mL

In an embodiment, a microinjection device is used to deliver a bisphosphonate to a subject at a dose of about 0.1 mg/day, or 0.2 mg/day, or 0.3 mg/day, or 0.4 mg/day, or 0.5 mg/day, or 0.6 mg/day, or 0.7 mg/day, or 0.8 mg/day, or 0.9 mg/day, or 1.0 mg/day, or 1.1 mg/day, or 1.2 mg/day, or 1.3 mg/day, or 1.4 mg/day, or 1.5 mg/day, or 1.6 mg/day, or 1.7 mg/day, or 1.8 mg/day, or 1.9 mg/day, or 2.0 mg/day, or 2.1 mg/day, or 2.2 mg/day, or 2.3 mg/day, or 2.4 mg/day, or 2.5 mg/day, or 2.6 mg/day, or 2.7 mg/day, or 2.8 mg/day, or 2.9 mg/day, or 3.0 mg/day, or 3.1 mg/day, or 3.2 mg/day, or 3.3 mg/day, or 3.4 mg/day, or 3.5 mg/day, or 3.6 mg/day, or 3.7 mg/day, or 3.8 mg/day, or 3.9 mg/day, or 4.0 mg/day, or 4.1 mg/day, or 4.2 mg/day, or 4.3 mg/day, or 4.4 mg/day, or 4.5 mg/day, or 4.6 mg/day, or 4.7 mg/day, or 4.8 mg/day, or 4.9 mg/day, or 5.0 mg/day.

In an embodiment, a microinjection device is used to deliver ibandronate to a subject at a dose of about 0.1 mg/day, or 0.2 mg/day, or 0.3 mg/day, or 0.4 mg/day, or 0.5 mg/day, or 0.6 mg/day, or 0.7 mg/day, or 0.8 mg/day, or 0.9 mg/day, or 1.0 mg/day, or 1.1 mg/day, or 1.2 mg/day, or 1.3 mg/day, or 1.4 mg/day, or 1.5 mg/day, or 1.6 mg/day, or 1.7 mg/day, or 1.8 mg/day, or 1.9 mg/day, or 2.0 mg/day, or 2.1 mg/day, or 2.2 mg/day, or 2.3 mg/day, or 2.4 mg/day, or 2.5 mg/day, or 2.6 mg/day, or 2.7 mg/day, or 2.8 mg/day, or 2.9 mg/day, or 3.0 mg/day, or 3.1 mg/day, or 3.2 mg/day, or 3.3 mg/day, or 3.4 mg/day, or 3.5 mg/day, or 3.6 mg/day, or 3.7 mg/day, or 3.8 mg/day, or 3.9 mg/day, or 4.0 mg/day, or 4.1 mg/day, or 4.2 mg/day, or 4.3 mg/day, or 4.4 mg/day, or 4.5 mg/day, or 4.6 mg/day, or 4.7 mg/day, or 4.8 mg/day, or 4.9 mg/day, or 5.0 mg/day.

In an embodiment, a microinjection device is used to deliver a bisphosphonate to a subject at a dose of about 0.1 mg or less, or 0.2 mg or less, or 0.3 mg or less, or 0.4 mg or less, or 0.5 mg or less, or 0.6 mg or less, or 0.7 mg or less, or 0.8 mg or less, or 0.9 mg or less, or 1.0 mg or less, or 1.1 mg or less, or 1.2 mg or less, or 1.3 mg or less, or 1.4 mg or less, or 1.5 mg or less, or 1.6 mg or less, or 1.7 mg or less, or 1.8 mg or less, or 1.9 mg or less, or 2.0 mg or less, or 2.1 mg or less, or 2.2 mg or less, or 2.3 mg or less, or 2.4 mg or less, or 2.5 mg or less, or 2.6 mg or less, or 2.7 mg or less, or 2.8 mg or less, or 2.9 mg or less, or 3.0 mg or less, or 3.1 mg or less, or 3.2 mg or less, or 3.3 mg or less, or 3.4 mg or less, or 3.5 mg or less, or 3.6 mg or less, or 3.7 mg or less, or 3.8 mg or less, or 3.9 mg or less, or 4.0 mg or less, or 4.1 mg or less, or 4.2 mg or less, or 4.3 mg or less, or 4.4 mg or less, or 4.5 mg or less, or 4.6 mg or less, or 4.7 mg or less, or 4.8 mg or less, or 4.9 mg or less, or 5.0 mg or less. In another embodiment, a microinjection device is used to deliver a bisphosphonate to a subject at a dose of at least about 1 mg, or 2 mg, or 3 mg on a daily basis. In another embodiment, a microinjection device is used to deliver a bisphosphonate to a subject once a day at a dosage of at least about 0.1 mg, or 0.5 mg, or 1 mg, or 2 mg, or 3 mg, or 4 mg, or 5 mg, or 6 mg, or 7 mg, or 8 mg, or 9 mg, or 10 mg, or more in a 3 mL formulation having bisphosphonate. In another embodiment, a microinjection device is used to deliver a bisphosphonate to a subject at a dosage of at least about 0.1 mg, or 0.5 mg, or 1 mg, or 2 mg, or 3 mg, or 4 mg, or 5 mg, or 6 mg, or 7 mg, or 8 mg, or 9 mg, or 10 mg, or more in an at least about 0.1 mL, or 1 mL, or 2 mL, or 3 mL or 4 mL, or 5 mL, or 6 mL, or 7 mL, or 8 mL, or 9 mL, or 10 mL, or more formulation having bisphosphonate.

In an embodiment, a microinjection device is used to deliver an ibandronate to a subject at a dose of about 0.1 mg or less, or 0.2 mg or less, or 0.3 mg or less, or 0.4 mg or less, or 0.5 mg or less, or 0.6 mg or less, or 0.7 mg or less, or 0.8 mg or less, or 0.9 mg or less, or 1.0 mg or less, or 1.1 mg or less, or 1.2 mg or less, or 1.3 mg or less, or 1.4 mg or less, or 1.5 mg or less, or 1.6 mg or less, or 1.7 mg or less, or 1.8 mg or less, or 1.9 mg or less, or 2.0 mg or less, or 2.1 mg or less, or 2.2 mg or less, or 2.3 mg or less, or 2.4 mg or less, or 2.5 mg or less, or 2.6 mg or less, or 2.7 mg or less, or 2.8 mg or less, or 2.9 mg or less, or 3.0 mg or less, or 3.1 mg or less, or 3.2 mg or less, or 3.3 mg or less, or 3.4 mg or less, or 3.5 mg or less, or 3.6 mg or less, or 3.7 mg or less, or 3.8 mg or less, or 3.9 mg or less, or 4.0 mg or less, or 4.1 mg or less, or 4.2 mg or less, or 4.3 mg or less, or 4.4 mg or less, or 4.5 mg or less, or 4.6 mg or less, or 4.7 mg or less, or 4.8 mg or less, or 4.9 mg or less, or 5.0 mg or less. In another embodiment, a microinjection device is used to deliver an ibandronate to a subject at a dose of at least about 1 mg, or 2 mg, or 3 mg on a daily basis. In another embodiment, a microinjection device is used to deliver an ibandronate to a subject once a day at a dosage of at least about 0.1 mg, or 0.5 mg, or 1 mg, or 2 mg, or 3 mg in a 3 mL formulation having ibandronate.

In an embodiment, the length of time in which a given dosage of a bisphosphonate is delivered to a subject using a microinjection device is dependent on various fluid and delivery properties, such as the volume of a bisphosphonate formulation, the viscosity of the formulation, the flow rate of the formulation from the microinjection device, the diameter of any fluid channels in any microneedles included in the microinjection device, and the pressure drop across fluid channels in any hollow microneedles included in the microinjection device. In an embodiment, a microinjection device can be used to deliver a bisphosphonate formulation to a subject in a time period between about 0.1 seconds and 60 minutes, or between about 30 seconds and 30 minutes, or between about 1 minute and 7 minutes, or between about 2 minutes and 6 minutes, or between about 3 minutes and 5 minutes. In another embodiment, a microinjection device can be used to deliver a bisphosphonate formulation to a subject in a time period up to an including 1 minute, or 2 minutes, or 3 minutes, or 4 minutes, or 5 minutes, or 6 minutes, or 7 minutes, or 8 minutes, or 9 minutes, or 10 minutes, or 30 minutes, or 1 hour, or 2 hours, or 3 hours, or 4 hours, or 5 hours, or 6 hours, or 7 hours, or 8 hours, or 9 hours, or 10 hours, or 15 hours, or 20 hours, or 24 hours.

In an embodiment, the length of time in which a given dosage of ibandronate is delivered to a subject using a microinjection device is dependent on various fluid and delivery properties, such as the volume of an ibandronate-containing formulation, the viscosity of the formulation, the flow rate of the formulation from the microinjection device, the diameter of any fluid channels in any microneedles included in the microinjection device, and the pressure drop across fluid channels in any microneedles included in the microinjection device. In an embodiment, a microinjection device can be used to deliver an ibandronate formulation to a subject in a time period between about 0.1 seconds and 60 minutes, or between about 30 seconds and 30 minutes, or between about 1 minute and 7 minutes, or between about 2 minutes and 6 minutes, or between about 3 minutes and 5 minutes. In another embodiment, a microinjection device can be used to deliver an ibandronate formulation to a subject in a time period up to an including 1 minute, or 2 minutes, or 3 minutes, or 4 minutes, or 5 minutes, or 6 minutes, or 7 minutes, or 8 minutes, or 9 minutes, or 10 minutes, or 30 minutes, or 1 hour, or 2 hours, or 3 hours, or 4 hours, or 5 hours, or 6 hours, or 7 hours, or 8 hours, or 9 hours, or 10 hours, or 15 hours, or 20 hours, or 24 hours.

In an embodiment, a microinjection device, such as any device provided herein, can be used to deliver a bisphosphonate, including ibandronate, to a subject at a dosage, in a 3.0 mL formulation of the bisphosphonate, of about 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg. In another embodiment, a microinjection device, such as any device provided herein, can be used to deliver a bisphosphonate to a subject every 1 hour, or 2 hours, or 3 hours, or 4 hours, or 5 hours, or 6 hours, or 7 hours, or 8 hours, or 9 hours, or 10 hours, or 11 hours, or 12 hours, or once a day. In another embodiment, a microinjection device, such as any device provided herein, can be used to deliver a bisphosphonate to a subject over a time period of about 0.1 seconds to 60 minutes, or about 1 second to 30 minutes, or about 5 seconds to 5 minutes, or about 10 seconds to 1 minute, or about 15 to 45 seconds. In an embodiment, a bisphosphonate can be delivered to a subject over a period of 1 minute or less, or 2 minutes or less, or 3 minutes or less, or 4 minutes or less, or 5 minutes or less, or 6 minutes or less, or 7 minutes or less, or 8 minutes or less, or 9 minutes or less, or 10 minutes or less, or 30 minutes or less, or 1 hour or less, or 2 hours or less, or 3 hours or less, or 4 hours or less, or 5 hours or less, or 6 hours or less, or 7 hours or less, or 8 hours or less, or 9 hours or less, or 10 hours or less, or 11 hours or less, or 12 hours or less, or 13 hours or less, or 14 hours or less, or 15 hours or less, or 16 hours or less, or 17 hours or less, or 18 hours or less, or 19 hours or less, or 20 hours or less, or 21 hours or less, or 22 hours or less, or 23 hours or less, or 24 hours or less. In another embodiment, a microinjection device, such as any device provided herein, can be used to deliver a bisphosphonate to a subject at a dosage of about 3.375 mg in a 3 mL formulation once a day over a period less than about 1 hour, or less than about 30 minutes, or less than about 10 minutes, or less than about 1 minute. In another embodiment, a microinjection device, such as any device provided herein, can be used to deliver a bisphosphonate to a subject in a time period of about 30 seconds, or 1 minute, or 1.5 minutes, or 2 minutes, or 2.5 minutes, or 3 minutes, or 3.5 minutes, or 4 minutes, or 4.5 minutes, or 5 minutes, or 5.5 minutes, or 6 minutes, or 7.5 minutes, or 8 minutes, or 8.5 minutes, or 9 minutes, or 9.5 minutes, or 10 minutes, or 10.5 minutes, or 11 minutes, or 11.5 minutes, or 12 minutes, or 12.5 minutes, or 13 minutes, or 13.5 minutes, or 14 minutes, or 14.5 minutes, or 15 minutes, or 15.5 minutes, or 16 minutes, or 16.5 minutes, or 17 minutes, or 17.5 minutes, or 18 minutes, or 18.5 minutes, or 19 minutes, or 19.5 minutes, or 20 minutes, or 20.5 minutes, or 21 minutes, or 21.5 minutes, or 22 minutes, or 22.5 minutes, or 23 minutes, or 23.5 minutes, or 24 minutes, or 24.5 minutes, or 25 minutes, or 25.5 minutes, or 26 minutes, or 26.5 minutes, or 27 minutes, or 27.5 minutes, or 28 minutes, or 28.5 minutes, or 29 minutes, or 29.5 minutes, or 30 minutes, or 30.5 minutes, or 31 minutes, or 31.5 minutes, or 32 minutes, or 32.5 minutes, or 33 minutes, or 33.5 minutes, or 34 minutes, or 34.5 minutes, or 35 minutes, or 35.5 minutes, or 36 minutes, or 36.5 minutes, or 37 minutes, or 37.5 minutes, or 38 minutes, or 38.5 minutes, or 39 minutes, or 39.5 minutes, or 40 minutes, or 40.5 minutes, or 41 minutes, or 41.5 minutes, or 42 minutes, or 42.5 minutes, or 43 minutes, or 43.5 minutes, or 44 minutes, or 44.5 minutes, or 45 minutes, or 45.5 minutes, or 46 minutes, or 46.5 minutes, or 47 minutes, or 47.5 minutes, or 48 minutes, or 48.5 minutes, or 49 minutes, or 49.5 minutes, or 50 minutes, or 50.5 minutes, or 51 minutes, or 51.5 minutes, or 52 minutes, or 52.5 minutes, or 53 minutes, or 53.5 minutes, or 54 minutes, or 54.5 minutes, or 55 minutes, or 55.5 minutes, or 56 minutes, or 56.5 minutes, or 57 minutes, or 57.5 minutes, or 58 minutes, or 58.5 minutes, or 59 minutes, or 59.5 minutes, or 60 minutes.

In an embodiment, a microinjection device, such as any device provided herein, can be used to deliver an ibandronate, including ibandronate, to a subject at a dosage, in a 3.0 mL formulation having ibandronate, of about 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg. In another embodiment, a microinjection device, such as any device provided herein, can be used to deliver ibandronate to a subject every 1 hour, or 2 hours, or 3 hours, or 4 hours, or 5 hours, or 6 hours, or 7 hours, or 8 hours, or 9 hours, or 10 hours, or 11 hours, or 12 hours, or once a day. In another embodiment, a microinjection device, such as any device provided herein, can be used to deliver ibandronate to a subject over a time period of about 0.1 seconds to 60 minutes, or about 1 second to 30 minutes, or about 5 seconds to 5 minutes, or about 10 seconds to 1 minute, or about 15 to 45 seconds. In an embodiment, ibandronate can be delivered to a subject over a period of 1 minute or less, or 2 minutes or less, or 3 minutes or less, or 4 minutes or less, or 5 minutes or less, or 6 minutes or less, or 7 minutes or less, or 8 minutes or less, or 9 minutes or less, or 10 minutes or less, or 30 minutes or less, or 1 hour or less, or 2 hours or less, or 3 hours or less, or 4 hours or less, or 5 hours or less, or 6 hours or less, or 7 hours or less, or 8 hours or less, or 9 hours or less, or 10 hours or less, or 11 hours or less, or 12 hours or less, or 13 hours or less, or 14 hours or less, or 15 hours or less, or 16 hours or less, or 17 hours or less, or 18 hours or less, or 19 hours or less, or 20 hours or less, or 21 hours or less, or 22 hours or less, or 23 hours or less, or 24 hours or less. In another embodiment, a microinjection device, such as any device provided herein, can be used to deliver ibandronate to a subject at a dosage of about 3.375 mg in a 3 mL formulation once a day over a period less than about 1 hour, or less than about 30 minutes, or less than about 10 minutes, or less than about 1 minute. In another embodiment, a microinjection device, such as any device provided herein, can be used to deliver ibandronate to a subject in a time period of about 30 seconds, or 1 minute, or 1.5 minutes, or 2 minutes, or 2.5 minutes, or 3 minutes, or 3.5 minutes, or 4 minutes, or 4.5 minutes, or 5 minutes, or 5.5 minutes, or 6 minutes, or 7.5 minutes, or 8 minutes, or 8.5 minutes, or 9 minutes, or 9.5 minutes, or 10 minutes, or 10.5 minutes, or 11 minutes, or 11.5 minutes, or 12 minutes, or 12.5 minutes, or 13 minutes, or 13.5 minutes, or 14 minutes, or 14.5 minutes, or 15 minutes, or 15.5 minutes, or 16 minutes, or 16.5 minutes, or 17 minutes, or 17.5 minutes, or 18 minutes, or 18.5 minutes, or 19 minutes, or 19.5 minutes, or 20 minutes, or 20.5 minutes, or 21 minutes, or 21.5 minutes, or 22 minutes, or 22.5 minutes, or 23 minutes, or 23.5 minutes, or 24 minutes, or 24.5 minutes, or 25 minutes, or 25.5 minutes, or 26 minutes, or 26.5 minutes, or 27 minutes, or 27.5 minutes, or 28 minutes, or 28.5 minutes, or 29 minutes, or 29.5 minutes, or 30 minutes, or 30.5 minutes, or 31 minutes, or 31.5 minutes, or 32 minutes, or 32.5 minutes, or 33 minutes, or 33.5 minutes, or 34 minutes, or 34.5 minutes, or 35 minutes, or 35.5 minutes, or 36 minutes, or 36.5 minutes, or 37 minutes, or 37.5 minutes, or 38 minutes, or 38.5 minutes, or 39 minutes, or 39.5 minutes, or 40 minutes, or 40.5 minutes, or 41 minutes, or 41.5 minutes, or 42 minutes, or 42.5 minutes, or 43 minutes, or 43.5 minutes, or 44 minutes, or 44.5 minutes, or 45 minutes, or 45.5 minutes, or 46 minutes, or 46.5 minutes, or 47 minutes, or 47.5 minutes, or 48 minutes, or 48.5 minutes, or 49 minutes, or 49.5 minutes, or 50 minutes, or 50.5 minutes, or 51 minutes, or 51.5 minutes, or 52 minutes, or 52.5 minutes, or 53 minutes, or 53.5 minutes, or 54 minutes, or 54.5 minutes, or 55 minutes, or 55.5 minutes, or 56 minutes, or 56.5 minutes, or 57 minutes, or 57.5 minutes, or 58 minutes, or 58.5 minutes, or 59 minutes, or 59.5 minutes, or 60 minutes.

In an embodiment, a microinjection device, such as any device provided herein, can be used to deliver a bisphosphonate to a subject once a day at a dose of about 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg, for a period up to an including 1 day, or 2 days, or 3 days, or 4 days, or 5 days, or 6 days, or 7 days, or 8 days, or 9 days, or 10 days, or 11 days, or 12 days, or 13 days, or 14 days, or 15 days, or 16 days, or 17 days, or 18 days, or 19 days, or 20 days, or 21 days, or 22 days, or 23 days, or 24 days, or 25 days, or 26 days, or 27 days, or 28 days, or 29 days, or 30 days, or 31 days. For example, a microinjection device can be used to deliver 3 mg of a bisphosphonate per dose to a subject once a day for up to 28 days. In another embodiment, a microinjection device, such as any device provided herein, can be used to deliver ibandronate to a subject once a day at a dose of about 0.1 mg, or 0.2 mg, or 0.3 mg, or 0.4 mg, or 0.5 mg, or 0.6 mg, or 0.7 mg, or 0.8 mg, or 0.9 mg, or 1.0 mg, or 1.1 mg, or 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.1 mg, or 3.2 mg, or 3.3 mg, or 3.4 mg, or 3.5 mg, or 3.6 mg, or 3.7 mg, or 3.8 mg, or 3.9 mg, or 4.0 mg, or 4.1 mg, or 4.2 mg, or 4.3 mg, or 4.4 mg, or 4.5 mg, or 4.6 mg, or 4.7 mg, or 4.8 mg, or 4.9 mg, or 5.0 mg, for a period up to an including 1 day, or 2 days, or 3 days, or 4 days, or 5 days, or 6 days, or 7 days, or 8 days, or 9 days, or 10 days, or 11 days, or 12 days, or 13 days, or 14 days, or 15 days, or 16 days, or 17 days, or 18 days, or 19 days, or 20 days, or 21 days, or 22 days, or 23 days, or 24 days, or 25 days, or 26 days, or 27 days, or 28 days, or 29 days, or 30 days, or 31 days. For example, a microinjection device can be used to deliver 3 mg of ibandronate per dose to a subject once a day for up to 28 days.

In an embodiment, the equivalent of at least about 1 mg, or 1.5 mg, or 2 mg, or 2.5 mg, or 3 mg, or 3.5 mg, or 4 mg of a bisphosphonate formulation in a 3 mL solution is delivered to a subject once a day. In another embodiment, the equivalent of at least about 1 mg, or 1.5 mg, or 2 mg, or 2.5 mg, or 3 mg, or 3.5 mg, or 4 mg of a bisphosphonate formulation in a 3 mL solution is delivered to a subject once a day and over a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. In an embodiment, a microinjection device, such as any device provided herein, is loaded with a bisphosphonate formulation having a bisphosphonate concentration of about 1 mg/3 mL, or 1.5 mg/3 mL, or 2.0 mg/3 mL, or 2.5 mg/3 mL, or 3 mg/3 mL, or 3.5 mg/3 mL, or 4 mg/3 mL, or 4.5 mg/3 mL, or 5 mg/3 mL. The microinjection device can be used to deliver the bisphosphonate formulation to a subject over a predetermined time period, such as a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. For example, a subject can apply a microinjection device having a bisphosphonate formulation with a bisphosphonate concentration of about 1 mg/3 mL, or 1.5 mg/3 mL, or 2.0 mg/3 mL, or 2.5 mg/3 mL, or 3 mg/3 mL, or 3.5 mg/3 mL, or 4 mg/3 mL, or 4.5 mg/3 mL, or 5 mg/3 mL to the subject's arm for delivery of bisphosphonate on a daily basis.

In an embodiment, the equivalent of at least about 1 mg, or 1.5 mg, or 2 mg, or 2.5 mg, or 3 mg, or 3.5 mg, or 4 mg of an ibandronate formulation in a 3 mL solution is delivered to a subject once a day. In another embodiment, the equivalent of at least about 1 mg, or 1.5 mg, or 2 mg, or 2.5 mg, or 3 mg, or 3.5 mg, or 4 mg of an ibandronate formulation in a 3 mL solution is delivered to a subject once a day and over a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. In an embodiment, a microinjection device, such as any device provided herein, is loaded with an ibandronate formulation having an ibandronate concentration of about 1 mg/3 mL, or 1.5 mg/3 mL, or 2.0 mg/3 mL, or 2.5 mg/3 mL, or 3 mg/3 mL, or 3.5 mg/3 mL, or 4 mg/3 mL, or 4.5 mg/3 mL, or 5 mg/3 mL. The microinjection device can be used to deliver the ibandronate formulation to a subject over a predetermined time period, such as a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. For example, a subject can apply a microinjection device having an ibandronate formulation with an ibandronate concentration of about 1 mg/3 mL, or 1.5 mg/3 mL, or 2.0 mg/3 mL, or 2.5 mg/3 mL, or 3 mg/3 mL, or 3.5 mg/3 mL, or 4 mg/3 mL, or 4.5 mg/3 mL, or 5 mg/3 mL to the subject's arm for delivery of ibandronate on a daily basis.

In an embodiment, a microinjection device having a deliverable bisphosphonate formulation is provided. A bisphosphonate, bisphosphonate-containing, ibandronate or ibandronate-containing formulation can be delivered by subcutaneous, transdermal or intradermal injection. A user places the device adjacent another user's skin or adjacent the user's skin, if self administration is desired, to deliver the bisphosphonate formulation. The user employs the microinjection device to deliver the bisphosphonate formulation to another user or the user (self administration). The user then removes the microinjection device from the skin. In an embodiment, the microinjection device is a single use device and is be disposed of after it is used. In another embodiment the microinjection device can be used for a future administration of the bisphosphonate formulation, such as with a replaceable cartridge or with additional doses provided in the original cartridge having the bisphosphonate formulation.

In an embodiment, bisphosphonate formulations can comprise inactive ingredients, such as one or more salts (e.g., sodium chloride), glacial acetic acid, sodium acetate and water. In another embodiment, a bisphosphonate formulation can comprise sodium chloride, glacial acetic acid, sodium acetate and water. In yet another embodiment, a formulation comprising ibandronate, ibandronate sodium, ibandronate monosodium salt monohydrate ibandronate, or other pharmaceutically acceptable salt of ibandronate can include inactive ingredients, such as one or more salts (e.g., sodium chloride), glacial acetic acid, sodium acetate and water. In still another embodiment, a formulation comprising ibandronate, ibandronate sodium, ibandronate monosodium salt monohydrate ibandronate, or other pharmaceutically acceptable salt of ibandronate can comprise sodium chloride, glacial acetic acid, sodium acetate and water.

In an embodiment, a microinjection device, such as any microinjection device provided herein, can be used to deliver a bisphosphonate formulation to a subject once a week, or twice a week, or three times a week, or four times a week, or five times a week, or six times a week, or seven times a week, or every other week, or two weeks in succession, or three weeks in succession. In an embodiment, a microinjection device, such as any microinjection device provided herein, can be used to deliver a bisphosphonate formulation to a subject at least once a week, twice a week, or three times a week, or at least once a month, twice a month, three times a month, or four times a month. In an embodiment, the bisphosphonate formulation can be administered every month, or every other month, or every third month, every fourth month, or every fifth month, or every sixth month. In an embodiment, on each day the bisphosphonate formulation is administered, it can be administered to a subject once a day, or twice a day, or three times a day, or four times a day, or five times a day, or six times a day, or seven times a day, or eight times a day, or nine times a day, or ten times a day.

In an embodiment, a microinjection device, such as any device provided herein, can be used to deliver a bisphosphonate to a subject at a dosage of about 1 mg, or 2 mg, or 3 mg, or 4 mg every 1 month, 2 months, 3 months or 4 months, and over a period of about 1 second to 10 minutes, or about 5 seconds to 5 minutes, or about 10 seconds to 1 minute, or about 15 to 30 seconds. In another embodiment, a microinjection device, such as any device provided herein, can be used to deliver a bisphosphonate to a subject at a dosage of about 3 mg every 3 months over a period of about 15 to 30 seconds.

In an embodiment, a microinjection device, such as any device provided herein, can be used to deliver ibandronate, ibandronate sodium, ibandronate monosodium salt monohydrate, or other pharmaceutically acceptable salt of ibandronate to a subject at a dosage of about 1 mg, or 2 mg, or 3 mg, or 4 mg every 1 month, 2 months, 3 months or 4 months, and over a period of about 1 second to 10 minutes, or about 5 seconds to 5 minutes, or about 10 seconds to 1 minute, or about 15 to 30 seconds. In another embodiment, a microinjection device, such as any device provided herein, can be used to deliver ibandronate, ibandronate sodium, ibandronate monosodium salt monohydrate, or other pharmaceutically acceptable salt of ibandronate to a subject at a dosage of about 3 mg every 3 months over a period of about 15 to 30 seconds.

In another embodiment, a microinjection device, such as any device provided herein, is used to deliver ibandronate sodium to a subject between once a year and 12 times a year, or between 3 times a year and 6 times a year. In an embodiment, ibandronate sodium is administered to a subject four times a year, such as every third month of the year.

In an embodiment, the time in which a given dosage of bisphosphonate is delivered to a subject a microinjection device is dependent on various fluid and delivery properties, such as the volume of bisphosphonate formulation, the viscosity of the formulation, the flow rate of the formulation from the microinjection device and the diameter of any fluid channels in any microneedles included in the microinjection device. In an embodiment, a microinjection device can be used to deliver a bisphosphonate formulation to a subject in a time period between about 1 second and 10 minutes, or between about 30 seconds and 8 minutes, or between about 1 minute and 7 minutes, or between about 2 minutes and 6 minutes, or between about 3 minutes and 5 minutes.

In an embodiment, a microinjection device having an ibandronate sodium-containing formulation is delivered to a subject four times a year, with a delivery time between about 3 minutes and 5 minutes.

In an embodiment, a microinjection device having a deliverable bisphosphonate formulation is provided. A user places the device adjacent another user's skin or adjacent the user's skin, if self administration is desired, to deliver the bisphosphonate formulation. The user employs the microinjection device to deliver the bisphosphonate formulation to another user or the user (self administration). The user then removes the microinjection device from the skin. In an embodiment, the microinjection device is a single use device and can be disposed of after it is used. In another embodiment the microinjection device can be used for a future administration of the bisphosphonate formulation, such as with a replaceable cartridge or with additional doses provided in the original cartridge having the bisphosphonate formulation.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1.-34. (canceled)

35. A system comprising a application device and a bisphosphonate formulation, the application device comprising:
a housing having a skin-contacting face defining an opening that can be positioned at a target site, said housing having a microneedle array; and
an impactor for impacting the microneedle array and accelerating the microneedle array toward the target site, the microneedle array configured to deliver a bisphosphonate formulation to the subject,
wherein the impactor is configured to move along a substantially arcuate path to move the microneedle array toward the target site.

36. The system of claim 35, wherein the bisphosphonate formulation comprises ibandronate, ibandronate sodium, ibandronate monosodium salt monohydrate, or other pharmaceutically acceptable salt of ibandronate.

37. The system of claim 35, wherein the bisphosphonate formulation has a bisphosphonate concentration between about 3.0 mg bisphosphonate in 3 mL and 3.5 mg in 3 mL.

38. The system of claim 37, wherein the bisphosphonate formulation has a bisphosphonate concentration between about 3.3 mg bisphosphonate in 3 mL and 3.4 mg in 3 mL.

39. The system of claim 35, wherein the bisphosphonate formulation has a pH between about 2.5 and 5.5.

40. The system of claim 39, wherein the bisphosphonate formulation has a pH between about 3.0 and 4.5.

41.-69. (canceled)

70. A system for applying a microneedle array to a subject's skin, comprising:
a bisphosphonate formulation;
a housing having a skin-contacting face defining an opening that can be positioned at a target site, said housing having a microneedle array; and
an impactor for impacting the microneedle array and accelerating the microneedle array toward the target site, the microneedle array configured to deliver a bisphosphonate formulation to the subject,
wherein the impactor is configured to move along a substantially arcuate path to move the microneedle array toward the target site.

71. The system of claim 70, wherein the bisphosphonate formulation comprises ibandronate, ibandronate sodium, ibandronate monosodium salt monohydrate, or other pharmaceutically acceptable salt of ibandronate.

72. The system of claim 70, wherein the bisphosphonate formulation has a bisphosphonate concentration between about 3.0 mg bisphosphonate in 3 mL and 3.5 mg in 3 mL.

73. The system of claim 72, wherein the bisphosphonate formulation has a bisphosphonate concentration between about 3.3 mg bisphosphonate in 3 mL and 3.4 mg in 3 mL.

74. The system of claim 70, wherein the bisphosphonate formulation has a pH between about 2.5 and 5.5.

75. The system of claim 74, wherein the bisphosphonate formulation has a pH between about 3.0 and 4.5.

76.-85. (canceled)

86. A system for delivering a bisphosphonate formulation to a subject, comprising:
a bisphosphonate formulation;
a microneedle array having one or more hollow tips for delivering the bisphosphonate formulation;
a housing having the microneedle array and a skin-contacting face defining an opening that can be positioned at or adjacent to a target site; and
a driver for moving the microneedle array toward the target site.

87. The system of claim 86, wherein the bisphosphonate formulation comprises ibandronate, ibandronate sodium, ibandronate monosodium salt monohydrate, or other pharmaceutically acceptable salt of ibandronate.

88. The system of claim 86, wherein the bisphosphonate formulation has a bisphosphonate concentration between about 3.0 mg bisphosphonate in 3 mL and 3.5 mg in 3 mL.

89. The system of claim 88, wherein the bisphosphonate formulation has a bisphosphonate concentration between about 3.3 mg bisphosphonate in 3 mL and 3.4 mg in 3 mL.

90. The system of claim 86, wherein the bisphosphonate formulation has a pH between about 2.5 and 5.5.

91. The system of claim 90, wherein the bisphosphonate formulation has a pH between about 3.0 and 4.5.

92.-93. (canceled)

94. The system of claim 86, wherein the bisphosphonate formulation comprises sodium chloride, glacial acetic acid, sodium acetate and water.

95.-125. (canceled)

126. The system of claim 35, wherein the bisphosphonate formulation comprises a pharmaceutically acceptable excipient.

127. The system of claim 70, wherein the bisphosphonate formulation comprises a pharmaceutically acceptable excipient.

128. The system of claim 86, wherein the bisphosphonate formulation comprises a pharmaceutically acceptable excipient.

129. A method for delivering a bisphosphonate formulation to a subject, comprising:
providing a system as in claim 86; and
delivering the bisphosphonate formulation to the subject with the aid of the microinjection device.

130. The method of claim 129, wherein the bisphosphonate formulation is delivered to the subject for a length of time between about 1 second and 10 minutes.

131. The method of claim 130, wherein the bisphosphonate formulation is delivered to the subject for a length of time between about 10 seconds and 1 minute.
